SEQUENCE LISTING (15 sequences)

SEQ ID NO: 1 - DNA, 16 bp, Artificial Sequence
  Description of Artifical Sequence Synthetic oligonucleotide
ctcaatccatggcagc

SEQ ID NO: 2 - DNA, 16 bp, Artificial Sequence
  Description of Artifical Sequence Synthetic oligonucleotide
tagcctgtcacttctc

SEQ ID NO: 3 - DNA, 17 bp, Artificial Sequence
  Description of Artifical Sequence Synthetic oligonucleotide
tagcttgtcccattctc

SEQ ID NO: 4 - DNA, 19 bp, Artificial Sequence
  Description of Artifical Sequence Synthetic oligonucleotide
tctcccagcgtgcgcccat

SEQ ID NO: 5 - DNA, 16 bp, Artificial Sequence
  Description of Artifical Sequence Synthetic oligonucleotide
tggcaagcatcctgta

SEQ ID NO: 6 - DNA, 21 bp, Artificial Sequence
  Description of Combined DNA/RNA Molecule Synthetic oligonucleotide
gcaugcggccucuguuugatt

SEQ ID NO: 7 - DNA, 21 bp, Artificial Sequence
  Description of Combined DNA/RNA Molecule Synthetic oligonucleotide
ucaaacagaggccgcaugctt

SEQ ID NO: 8 - protein, 12 aa, Artificial Sequence
  Description of Artifical Sequence Synthetic peptide
CYGRKKRRQRRR

SEQ ID NO: 9 - protein, 10 aa, Artificial Sequence
  Description of Artifical Sequence Synthetic peptide
CRRRRRRRRR

SEQ ID NO: 10 - protein, 13 aa, Artificial Sequence
  Description of Artifical Sequence Synthetic peptide
CYGRKKRRQRRRC

SEQ ID NO: 11 - protein, 23 aa, Artificial Sequence
  Description of Artifical Sequence Synthetic peptide
RRRQRRKKRGYRRRQRRKKRGYC

SEQ ID NO: 12 - protein, 4 aa, Artificial Sequence
  Description of Artifical Sequence Synthetic peptide
RGDC

SEQ ID NO: 13 - protein, 5 aa, Artificial Sequence
  Description of Artifical Sequence Synthetic peptide
RGDFC

SEQ ID NO: 14 - protein, 19 aa, Artificial Sequence
  Description of Artifical Sequence Synthetic peptide
SDGRGGGRRRQRRKKRGYC

SEQ ID NO: 15 - protein, 9 aa, Artificial Sequence
  Description of Artifical Sequence Synthetic peptide
RRRRRRRRR

(19) United States
(12) Patent Application Publication
(10) Pub. No.: US 2011/0111044 A1
(43) Pub. Date: May 12, 2011

(54) NANOPARTICLE COMPOSITIONS FOR NUCLEIC ACIDS DELIVERY SYSTEM

(75) Inventors: Hong Zhao, Edison, NJ (US); Lianjun Shi, Bridgewater, NJ (US)

(73) Assignee: ENZON PHARMACEUTICALS, INC., Bridgewater, NJ (US)

(21) Appl. No.: 13/003,816

(22) PCT Filed: Jul. 31, 2009
(86) PCT No.: PCT/US09/52396

§ 371 Date: Jan. 12, 2011

Related U.S. Application Data
(60) Provisional application No. 61/085,289, filed on Jul. 31, 2008.

(51) Int. Cl. A61K 9/50; C12N 15/88; C12N 5/071; A61K 31/7088; C12N 5/09; A61K 31/713; A61K 31/711; A61K 31/7105; A61K 38/02; A61P 35/00; A61P 29/00; A61P 31/12; A61K 47/44; B82Y 5/00

(52) U.S. Cl. 424/502; 435/458; 435/375; 514/44 A; 514/44 R; 514/1.1; 514/785; 977/773; 977/906

(57) ABSTRACT

The present invention is directed to nanoparticle compositions for the delivery of oligonucleotides and methods of modulating an expression of a targeted gene using the nanoparticle compositions. In particular, the invention relates to oligonucleotides encapsulated in a mixture of a cationic lipid, a fusogenic lipid and a PEG lipid.

### CROSS-REFERENCE TO RELATED APPLICATION

This application claims the benefit of priority from U.S. Provisional Patent Application Ser. No. 61/085,289 filed Jul. 31, 2008, the contents of which are incorporated herein by reference.

### FIELD OF THE INVENTION

The present invention relates to nanoparticle compositions for the delivery of oligonucleotides and methods of modulating gene expression using nanoparticle compositions.

### BACKGROUND OF THE INVENTION

Therapy using nucleic acids has been proposed as an endeavor to treat various diseases over the past years. Therapy such as antisense therapy is a powerful tool in the treatment of disease because a therapeutic gene can selectively modulate gene expression associated with disease and minimize side effects which occur when other therapeutic approaches are used.

Therapy using nucleic acids has, however, been limited due to poor stability of genes and ineffective delivery. Several gene delivery systems have been proposed to overcome the hurdles and effectively introduce therapeutic genes into a targeted area, such as cancer cells or tissues in vitro and in vivo. Such attempts to improve delivery and enhance cellular uptake of therapeutic genes are directed to utilizing liposomes.

Currently available liposomes do not effectively deliver oligonucleotides into the body, although some progress has been made in the delivery of plasmids. In the delivery of oligonucleotides, desirable delivery systems should include positive charges sufficient enough to neutralize the negative charges of oligonucleotides. Recently, coated cationic liposomal (CCL) and Stable Nucleic Acid-Lipid Particles (SNALP) formulations described by Stuart, D. D., et al Biochim. Biophys. Acta, 2000, 1463:219-229 and Semple, S. C., et al, Biochim. Biophys. Acta, 2001, 1510:152-166, respectively, were reported to provide nanoparticles with small sizes, high nucleic acid encapsulation rate, good serum stability, and long circulation time. However, they did not show significantly improved in vivo activities especially in organs other than the liver, as compared to the use of the naked oligonucleotides. It is desirable to provide a nucleic acids delivery system which allows enhanced cellular uptake and increased bioavailability of oligonucleotides in the cells, e.g. cancer cells. It is also desirable if the nucleic acids delivery system is stable for storage and safe for clinical use.

In spite of the attempts and advances, there continues to be a need to provide improved nucleic acids delivery systems. The present invention addresses this need.

### SUMMARY OF THE INVENTION

The present invention provides nanoparticle compositions for nucleic acids delivery. Nucleic acids, such as oligonucleotides, are encapsulated within nanoparticle complexes containing a mixture of a cationic lipid, a fusogenic lipid and a PEG lipid.

In accordance with this aspect of the invention, the nanoparticle composition for the delivery of nucleic acids (i.e., an oligonucleotide) includes:

(i) a cationic lipid of Formula (I):

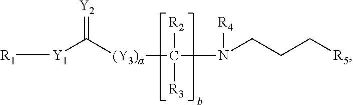

wherein
R₁ is a cholesterol or analog thereof;
Y₁ and Y₃ are independently O, S or NR₇, preferably O or S and more preferably O;
Y₂ is O, S or NR₇, preferably O or S and more preferably O;
(a) is 0 or 1;
R₂ and R₃ are independently hydrogen or lower alkyl;
(b) is a positive integer from about 2 to about 10 (i.e., 2, 3, 4, 5, 6, 7, 8, 9 and 10, preferably 2);
R₄ is hydrogen, lower alkyl or

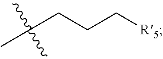

R₅ is

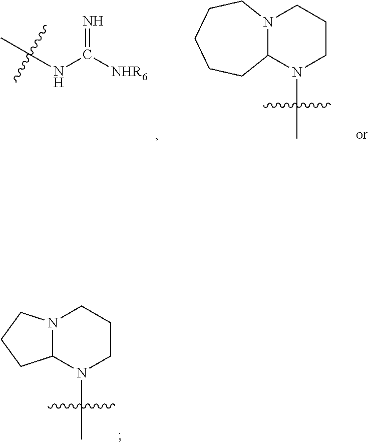

R′₅ is NH₂,

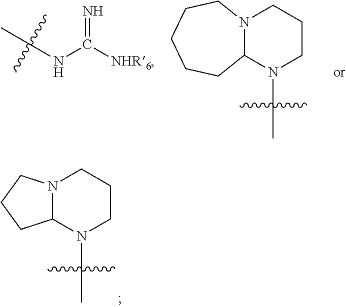

and
R₆, R′₆ and R₇ are independently hydrogen or lower alkyl,

(ii) a fusogenic lipid; and

(iii) a PEG lipid.

The present invention also provides methods for the delivery of nucleic acids (preferably oligonucleotides) to a cell or tissue, in vivo and in vitro. Oligonucleotides introduced by the methods described herein can modulate expression of a target gene.

One preferred aspect of the present invention provides methods of inhibiting expression of a target gene, i.e., oncogenes and genes associated with inflammation in mammals, preferably humans. The methods include contacting cells such as cancer cells or tissues with a nanoparticle prepared from the nanoparticle composition described herein. The oligonucleotides encapsulated within the nanoparticle are released and mediate down-regulation of mRNA or protein in the cells or tissues being treated. The treatment with the nanoparticle allows modulation of target gene expression and the attendant benefits associated therewith in the treatment of malignant disease, such as inhibition of the growth of cancer cells. Such therapies can be carried out as a single treatment or as a part of combination therapy, with one or more useful and/or approved treatments.

Further aspects include methods of making the cationic lipids of Formula (I) as well as nanoparticles containing the same.

One advantage of the present invention is that the nanoparticle compositions containing a cationic lipid described herein provide a means for in vivo as well as in vitro administration of nucleic acids. This delivery technology allows enhanced stability, transfection efficiency, and bioavailability of therapeutic oligonucleotides in the body, thus allowing the artisan to achieve a desired therapeutic efficacy of oligonucleotides.

The nanoparticles described herein have improved in vitro cellular uptake of LNA-containing oligonucleotides in human cancer cells and enhanced the delivery of LNA-ONs to the tumors in mammals.

The cationic lipids described herein neutralize the negative charges of nucleic acids and facilitate cellular uptake of the nanoparticle containing the nucleic acids therein. The cationic lipids herein further provide multiple units of cationic moieties per cholesterol moiety, to provide higher efficiency in (i) neutralizing the negative charges of the nucleic acids and (ii) forming a tighter ionic complex with nucleic acids. This technology is advantageous for the delivery of therapeutic oligonucleotides and the treatment of mammals, i.e., humans, using therapeutic oligonucleotides including LNA, and those based on siRNA, microRNA, and MOE antisense.

Another advantage of the cationic lipids described herein is that they provide a means to control the size of the nanoparticles by forming multiple ionic complexes with nucleic acids.

The cationic lipids described herein stabilize nanoparticle complexes and nucleic acids therein in biological fluids. Without being bound by any theory, it is believed that the nanoparticle complex enhances the stability of the encapsulated nucleic acids at least in part by shielding the molecules from nucleases, thereby protecting from degradation. The nanoparticles based on cationic lipids of Formula (I) described herein stabilize the encapsulated nucleic acids.

The cationic lipids described herein allow high efficiency (e.g. above 70%, preferably above 80%) of nucleic acids (oligonucleotides) loading compared to art-known neutral or negatively charged nanoparticles, which typically have loadings of about or less than 10%. Without being bound by any theory, the high loading is achieved in part by the fact that the guanidinium group having high pKa (13-14) of the cationic lipids of Formula (I) described herein forms substantially compact zwitter ionic hydrogen bonds with phosphate groups of nucleic acids, thereby enabling more nucleic acids to be effectively packaged into the inner compartment of nanoparticles.

The nanoparticles described herein provide a further advantage over neutral or negatively charged nanoparticles, in that the aggregation or precipitation of nanoparticles is less likely to occur. Without being bound by any theory, the desired property is attributed in part to the fact that the cationic lipids forming hydrogen bonds or electrostatic interaction with nucleic acids are encapsulated within the nanoparticles, and noncationic/fusogenic lipids and PEG lipids surround the cationic lipid and nucleic acids.

The nanoparticles described herein provide another advantage, such as higher transfection efficiency. The nanoparticles described herein allow transfection of cells in vitro and in vivo without an aid of a transfecting agent. The nanoparticles are safe, because they do not have the same toxicity as art-known nanoparticles, which require transfecting agents. The higher transfection efficiency of the nanoparticles also provides a means to deliver therapeutic nucleic acids into a nucleus.

The nanoparticles described herein also provide an advantageous stability and flexibility in the preparation of the nanoparticles. The nanoparticles can be prepared in a wide pH range, such as 2-12. The nanoparticles described herein also can be used clinically at a desirable physiological pH, such as 7.2-7.6.

The nanoparticle delivery systems described herein also allow sufficient amounts of the therapeutic oligonucleotides to be selectively available at the desired target area such as cancer cells via EPR effects. The nanoparticle composition described herein thus improves specific mRNA down regulation in cancer cells or tissues.

Another advantage is that the cationic lipids described herein allow for the preparation of homogenous nanoparticles in size and stability of the nanoparticles during storage. The nanoparticle complexes containing the cationic lipids described herein are stable under buffer conditions. This is a significant advantage over prior art technologies since this feature provides clinicians with reliable and flexible treatment regimens. The stable nanoparticles are suitable for the systemic delivery of LNA-ON.

Another advantage is that the nanoparticles described herein allow delivery of one or more different target oligonucleotides, thereby attaining synergistic effects in treatment of disease.

It has been increasingly attractive to treat human diseases at the gene level. Oligonucleotides, including locked nucleic acids and siRNA, have the potential to prohibit unwanted gene expression. The present invention allows for enhancement in cellular uptake and accumulation of nucleic acids such as LNA-ONs in the target area, cells or tissues. In addition, the cationic lipid-based nanoparticles described herein are safe to deliver oligonucleotides in vivo to improve their pharmacokinetic profile, cell penetration, and specific tumor targeting, as compared to viral delivery systems.

Another advantage of the present invention is that the nanoparticle described herein enables potent down-modulation of target mRNA in multiple human tumor cells without an aid of transfection agents and improves the cellular delivery of nucleic acids in tumor-bearing mammals. When given intravenously, the oligonucleotides encapsulated in the nanoparticles are >30-fold and >3-fold more effective than naked oligonucleotides on silencing mRNA in the livers and tumors, respectively.

Other and further advantages will be apparent from the following description.

For purposes of the present invention, the term “residue” shall be understood to mean that portion of a compound, to which it refers, e.g., cholesterol, etc. that remains after it has undergone a substitution reaction with another compound.

For purposes of the present invention, the term “alkyl” refers to a saturated aliphatic hydrocarbon, including straight-chain, branched-chain, and cyclic alkyl groups. The term “alkyl” also includes alkyl-thio-alkyl, alkoxyalkyl, cycloalkylalkyl, heterocycloalkyl, and C₁₋₆ alkylcarbonylalkyl groups. Preferably, the alkyl group has 1 to 12 carbons. More preferably, it is a lower alkyl of from about 1 to 7 carbons, yet more preferably about 1 to 4 carbons. The alkyl group can be substituted or unsubstituted. When substituted, the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “substituted” refers to adding or replacing one or more atoms contained within a functional group or compound with one of the moieties from the group of halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ alkylcarbonylalkyl, aryl, and amino groups.

For purposes of the present invention, the term “alkenyl” refers to groups containing at least one carbon-carbon double bond, including straight-chain, branched-chain, and cyclic groups. Preferably, the alkenyl group has about 2 to 12 carbons. More preferably, it is a lower alkenyl of from about 2 to 7 carbons, yet more preferably about 2 to 4 carbons. The alkenyl group can be substituted or unsubstituted. When substituted the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups.

For purposes of the present invention, the term “alkynyl” refers to groups containing at least one carbon-carbon triple bond, including straight-chain, branched-chain, and cyclic groups. Preferably, the alkynyl group has about 2 to 12 carbons. More preferably, it is a lower alkynyl of from about 2 to 7 carbons, yet more preferably about 2 to 4 carbons. The alkynyl group can be substituted or unsubstituted. When substituted the substituted group(s) preferably include halo, oxy, azido, nitro, cyano, alkyl, alkoxy, alkyl-thio, alkyl-thio-alkyl, alkoxyalkyl, alkylamino, trihalomethyl, hydroxyl, mercapto, hydroxy, cyano, alkylsilyl, cycloalkyl, cycloalkylalkyl, heterocycloalkyl, heteroaryl, alkenyl, alkynyl, C₁₋₆ hydrocarbonyl, aryl, and amino groups. Examples of “alkynyl” include propargyl, propyne, and 3-hexyne.

For purposes of the present invention, the term “aryl” refers to an aromatic hydrocarbon ring system containing at least one aromatic ring. The aromatic ring can optionally be fused or otherwise attached to other aromatic hydrocarbon rings or non-aromatic hydrocarbon rings. Examples of aryl groups include, for example, phenyl, naphthyl, 1,2,3,4-tetrahydronaphthalene and biphenyl. Preferred examples of aryl groups include phenyl and naphthyl.

For purposes of the present invention, the term “cycloalkyl” refers to a C₃₋₈ cyclic hydrocarbon. Examples of cycloalkyl include cyclopropyl, cyclobutyl, cyclopentyl, cyclohexyl, cycloheptyl and cyclooctyl.

For purposes of the present invention, the term “cycloalkenyl” refers to a C₃₋₈ cyclic hydrocarbon containing at least one carbon-carbon double bond. Examples of cycloalkenyl include cyclopentenyl, cyclopentadienyl, cyclohexenyl, 1,3-cyclohexadienyl, cycloheptenyl, cycloheptatrienyl, and cyclooctenyl.

For purposes of the present invention, the term “cycloalkylalkyl” refers to an alklyl group substituted with a C₃₋₈ cycloalkyl group. Examples of cycloalkylalkyl groups include cyclopropylmethyl and cyclopentylethyl.

For purposes of the present invention, the term “alkoxy” refers to an alkyl group of indicated number of carbon atoms attached to the parent molecular moiety through an oxygen bridge. Examples of alkoxy groups include, for example, methoxy, ethoxy, propoxy and isopropoxy.

For purposes of the present invention, an “alkylaryl” group refers to an aryl group substituted with an alkyl group.

For purposes of the present invention, an “aralkyl” group refers to an alkyl group substituted with an aryl group.

For purposes of the present invention, the term “alkoxyalkyl” group refers to an alkyl group substituted with an alkloxy group.

For purposes of the present invention, the term “alkyl-thio-alkyl” refers to an alkyl-S-alkyl thioether, for example methylthiomethyl or methylthioethyl.

For purposes of the present invention, the term “amino” refers to a nitrogen containing group as is known in the art derived from ammonia by the replacement of one or more hydrogen radicals by organic radicals. For example, the terms “acylamino” and “alkylamino” refer to specific N-substituted organic radicals with acyl and alkyl substituent groups respectively.

For purposes of the present invention, the term “alkylcarbonyl” refers to a carbonyl group substituted with alkyl group.

For purposes of the present invention, the term “halogen’ or “halo” refers to fluorine, chlorine, bromine, and iodine.

For purposes of the present invention, the term “heterocycloalkyl” refers to a non-aromatic ring system containing at least one heteroatom selected from nitrogen, oxygen, and sulfur. The heterocycloalkyl ring can be optionally fused to or otherwise attached to other heterocycloalkyl rings and/or non-aromatic hydrocarbon rings. Preferred heterocycloalkyl groups have from 3 to 7 members. Examples of heterocycloalkyl groups include, for example, piperazine, morpholine, piperidine, tetrahydrofuran, pyrrolidine, and pyrazole. Preferred heterocycloalkyl groups include piperidinyl, piperazinyl, morpholinyl, and pyrrolidinyl.

For purposes of the present invention, the term “heteroaryl” refers to an aromatic ring system containing at least one heteroatom selected from nitrogen, oxygen, and sulfur. The heteroaryl ring can be fused or otherwise attached to one or more heteroaryl rings, aromatic or non-aromatic hydrocarbon rings or heterocycloalkyl rings. Examples of heteroaryl groups include, for example, pyridine, furan, thiophene, 5,6,7,8-tetrahydroisoquinoline and pyrimidine. Preferred examples of heteroaryl groups include thienyl, benzothienyl, pyridyl, quinolyl, pyrazinyl, pyrimidyl, imidazolyl, benzimidazolyl, furanyl, benzofuranyl, thiazolyl, benzothiazolyl, isoxazolyl, oxadiazolyl, isothiazolyl, benzisothiazolyl, triazolyl, tetrazolyl, pyrrolyl, indolyl, pyrazolyl, and benzopyrazolyl.

For purposes of the present invention, the term “heteroatom” refers to nitrogen, oxygen, and sulfur.

In some embodiments, substituted alkyls include carboxyalkyls, aminoalkyls, dialkylaminos, hydroxyalkyls and mercaptoalkyls; substituted alkenyls include carboxyalkenyls, aminoalkenyls, dialkenylaminos, hydroxyalkenyls and mercaptoalkenyls; substituted alkynyls include carboxyalkynyls, aminoalkynyls, dialkynylaminos, hydroxyalkynyls and mercaptoalkynyls; substituted cycloalkyls include moieties such as 4-chlorocyclohexyl; aryls include moieties such as napthyl; substituted aryls include moieties such as 3-bromo phenyl; aralkyls include moieties such as tolyl; heteroalkyls include moieties such as ethylthiophene; substituted heteroalkyls include moieties such as 3-methoxy-thiophene; alkoxy includes moieties such as methoxy; and phenoxy includes moieties such as 3-nitrophenoxy. Halo shall be understood to include fluoro, chloro, iodo and bromo.

For purposes of the present invention, “positive integer” shall be understood to include an integer equal to or greater than 1 and as will be understood by those of ordinary skill to be within the realm of reasonableness by the artisan of ordinary skill.

For purposes of the present invention, the term “linked” shall be understood to include covalent (preferably) or noncovalent attachment of one group to another, i.e., as a result of a chemical reaction.

The terms “effective amounts” and “sufficient amounts” for purposes of the present invention shall mean an amount which achieves a desired effect or therapeutic effect as such effect is understood by those of ordinary skill in the art.

The term “nanoparticle” and/or “nanoparticle complex” formed using the nanoparticle composition described herein refers to a lipid-based nanocomplex. The nanoparticle contains nucleic acids such as oligonucleotides encapsulated in a mixture of a cationic lipid, a fusogenic lipid, and a PEG lipid. Alternatively, the nanoparticle can be formed without nucleic acids.

For purposes of the present invention, the term “therapeutic oligonucleotide” refers to an oligonucleotide used as a pharmaceutical or diagnostic agent.

For purposes of the present invention, “modulation of gene expression” shall be understood as broadly including down-regulation or up-regulation of any types of genes, preferably associated with cancer and inflammation, compared to a gene expression observed in the absence of the treatment with the nanoparticle described herein, regardless of the route of administration.

For purposes of the present invention, “inhibition of expression of a target gene” shall be understood to mean that mRNA expression or the amount of protein translated are reduced or attenuated when compared to that observed in the absence of the treatment with the nanoparticle described herein. Suitable assays of such inhibition include, e.g., examination of protein or mRNA levels using techniques known to those of skill in the art such as dot blots, northern blots, in situ hybridization, ELISA, immunoprecipitation, enzyme function, as well as phenotypic assays known to those of skill in the art. The treated conditions can be confirmed by, for example, decrease in mRNA levels in cells, preferably cancer cells or tissues.

Broadly speaking, successful inhibition or treatment shall be deemed to occur when the desired response is obtained. For example, successful inhibition or treatment can be defined by obtaining e.g, 10% or higher (i.e. 20% 30%, 40%) downregulation of genes associated with tumor growth inhibition. Alternatively, successful treatment can be defined by obtaining at least 20% or preferably 30%, more preferably 40% or higher (i.e., 50% or 80%) decrease in oncogene mRNA levels or encoded protein levels in cancer cells or tissues, including other clinical markers contemplated by the artisan in the field, when compared to that observed in the absence of the treatment with the nanoparticle described herein.

Further, the use of singular terms for convenience in description is in no way intended to be so limiting. Thus, for example, reference to a composition comprising an oligonucleotide, a cholesterol analog, a fusogenic lipid, a PEG lipid etc. refers to one or more molecules of that oligonucleotide, cholesterol analog, fusogenic lipid, PEG lipid, etc. It is also contemplated that the oligonucleotide can be the same or different kind of gene. It is also to be understood that this invention is not limited to the particular compositions, process steps, and materials disclosed herein as such compositions, process steps, and materials may vary somewhat.

It is also to be understood that the terminology employed herein is used for the purpose of describing particular embodiments only and is not intended to be limiting, since the scope of the present invention will be limited by the appended claims and equivalents thereof.

### BRIEF DESCRIPTION OF THE DRAWINGS

FIG. 1 schematically illustrates a reaction scheme of preparing 2-[bis(3-guanidiniumpropyl)]aminoethylcholesteryl carbonate (compound 5), as described in Examples 1-5.

FIG. 2 describes the stability of nanoparticles as described in Example 7.

FIG. 3 describes the cellular uptake and intracellular distribution of nanoparticles encapsulating nucleic acids, as described in Example 8.

FIG. 4 describes the in vitro efficacy of nanoparticles on ErbB3 expression in human epidermal cancer cells, as described in Example 9.

FIG. 5 describes the in vitro efficacy of nanoparticles on ErbB3 expression in human gastric cancer cells, as described in Example 10.

FIG. 6 describes the in vitro efficacy of nanoparticles on ErbB3 expression in human lung cancer cells, as described in Example 11.

FIG. 7 describes the in vitro efficacy of nanoparticles on ErbB3 expression in human prostate cancer cells, as described in Example 12.

FIG. 8 describes the in vitro efficacy of nanoparticles on ErbB3 expression in human breast cancer cells, as described in Example 13.

FIG. 9 describes the in vitro efficacy of nanoparticles on ErbB3 expression in human KB cancer cells, as described in Example 14.

FIG. 10 describes the in vitro efficacy of nanoparticles on ErbB3 expression in human prostate cancer cells, as described in Example 15.

FIG. 11 describes the in vivo efficacy of nanoparticles on ErbB3 expression in the tumors of human prostate cancer xenografted mice, as described in Example 16.

FIG. 12 describes the in vivo efficacy of nanoparticles on ErbB3 expression in the livers of human prostate cancer xenografted mice, as described in Example 16.

FIG. 13 describes the in vivo efficacy of nanoparticles on ErbB3 expression in the tumor of human colon cancer xenografted mice, as described in Example 17.

FIG. 14 describes the in vivo efficacy of nanoparticles on ErbB3 expression in human cancer xenografted mice with metastasis in liver, as described in Example 18.

### DETAILED DESCRIPTION OF THE INVENTION

### A. Overview

In one aspect of the present invention, there are provided nanoparticle compositions for the delivery of nucleic acids. The nanoparticle composition contains (i) a cationic lipid; (ii) a fusogenic lipid; and (iii) a PEG lipid. The nucleic acids contemplated include oligonucleotides or plasmids, and preferably oligonucleotides. The nanoparticles prepared by using the nanoparticle composition described herein include nucleic acids encapsulated in the lipid carrier.

### B. Cationic Lipids

The nanoparticle composition described herein contains a cationic lipid of Formula (I):

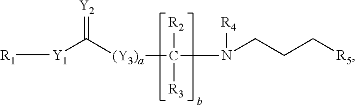

wherein
R₁ is a cholesterol or analog thereof;
Y₁ and Y₃ are independently O, S or NR₇, preferably O or S and more preferably O;
Y₂ is O, S or NR₇, preferably O or S and more preferably O;
(a) is 0 or 1;
R₂ and R₃ are independently selected hydrogen or lower alkyls such as C₁₋₇ alkyls, preferably hydrogen or C₁₋₄ alkyls;
(b) is a positive integer from about 2 to about 10 (i.e., 2, 3, 4, 5, 6, 7, 8, 9, 10 and in some embodiments, preferably 2, 3, 4, more preferably 2);
R₄ is hydrogen, lower alkyls such as C₁₋₇ alkyls (i.e., C₁₋₄ alkyls) or

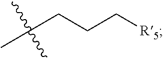

R₅ is

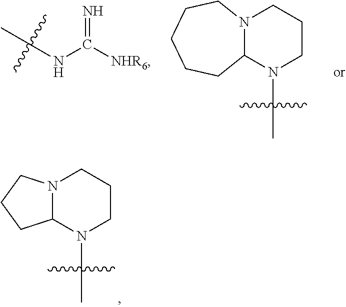

preferably

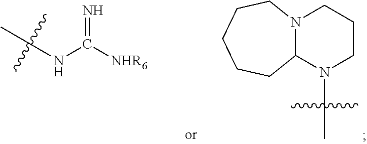

R′₅ is NH₂,

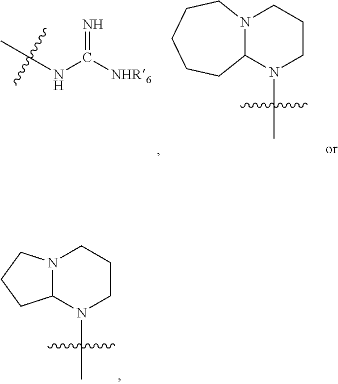

preferably

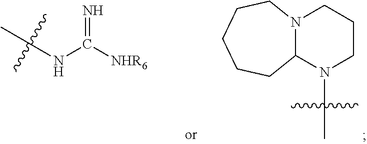

and
R₆, R′₆ and R₇ are independently selected hydrogen or lower alkyls such as C₁₋₇ alkyls, preferably hydrogen or C₁₋₄ alkyls.

For purposes of the present invention, C(R₂)(R₃) is the same or different when (b) is equal to or greater than 2.

In one preferred aspect of the invention, the cationic lipid described herein includes more than one (i.e. two) moieties containing positively charged groups.

In another preferred aspect, the cationic lipid includes each R₅ and R′₅ containing the structure of:

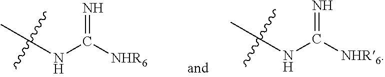

wherein both R₆ and R′₆ are preferably hydrogen. The cationic lipid preferably has two units of a guanidinylpropyl group such as

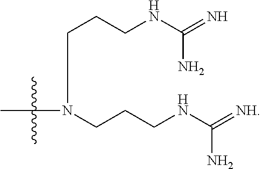

In yet another preferred aspect, Y₁, Y₂ and Y₃ of Formula (I) are all oxygen.

In yet another preferred aspect of the cationic lipid, (a) is 1 and (b) is 2.

In yet another preferred aspect of the cationic lipid, both R₂ and R₃ are hydrogen.

The cationic lipids of Formula (I) described herein carry a net positive charge at a selected pH such as pH<13 (e.g. pH 6-12, pH 6-8).

In one particular embodiment, the nanoparticle compositions described herein include the cationic lipids having the structure:

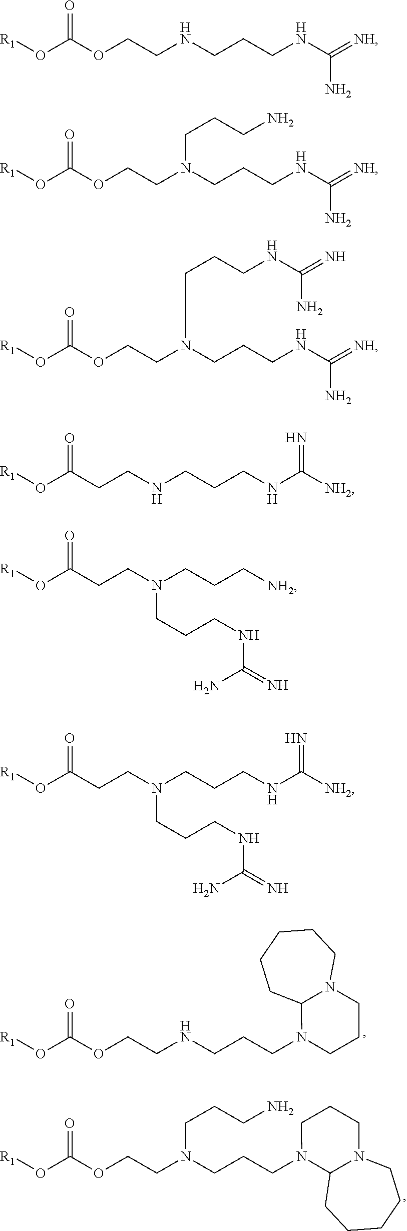

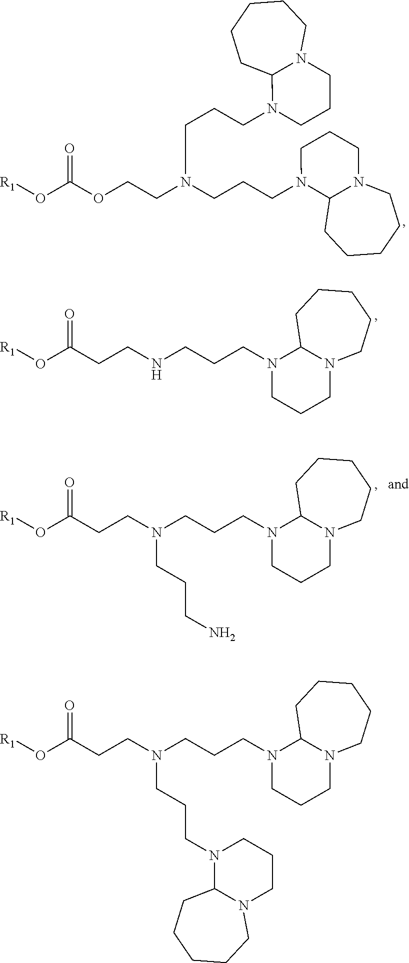

wherein, R₁ is cholesterol or an analog thereof.

Preferably, the nanoparticle compositions described herein include the cationic lipids having the structure:

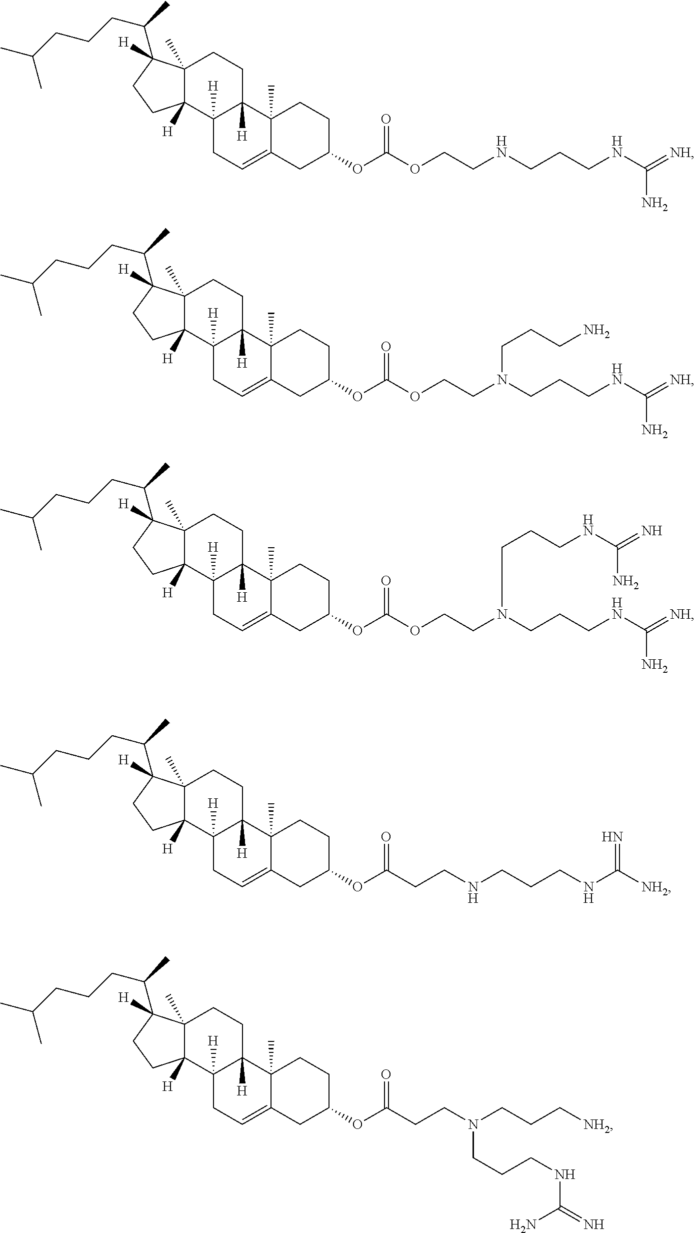

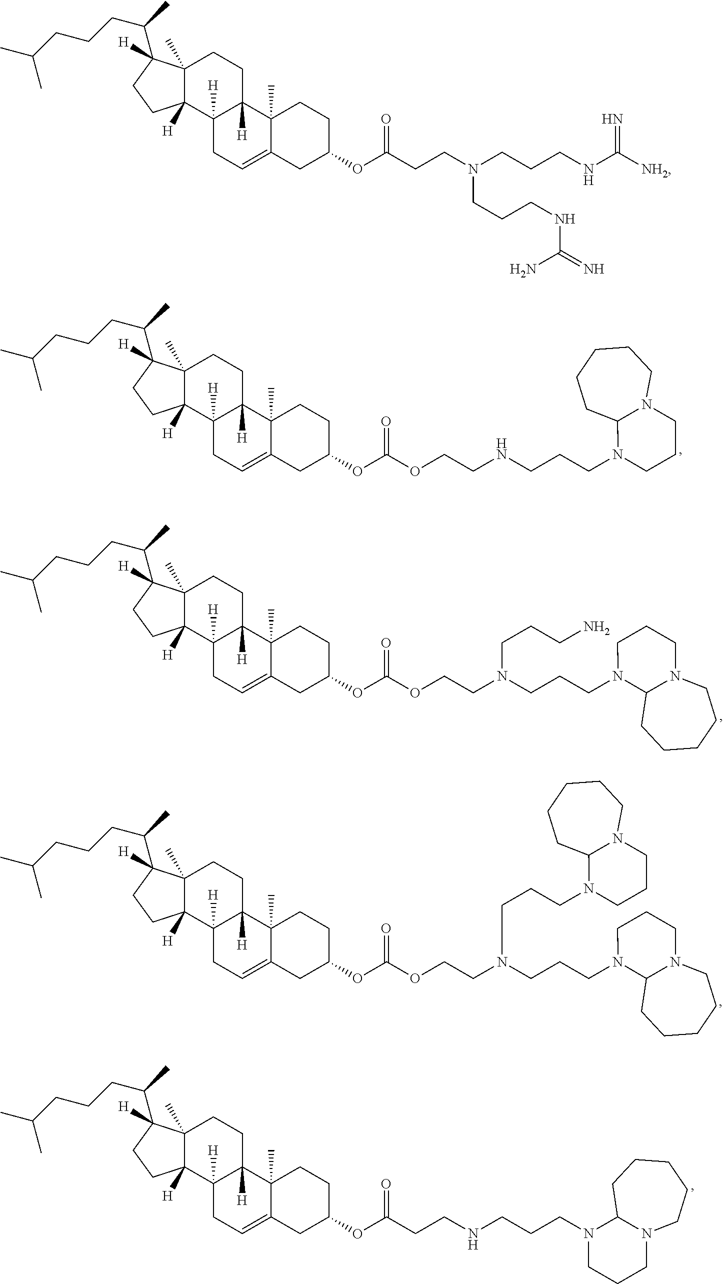

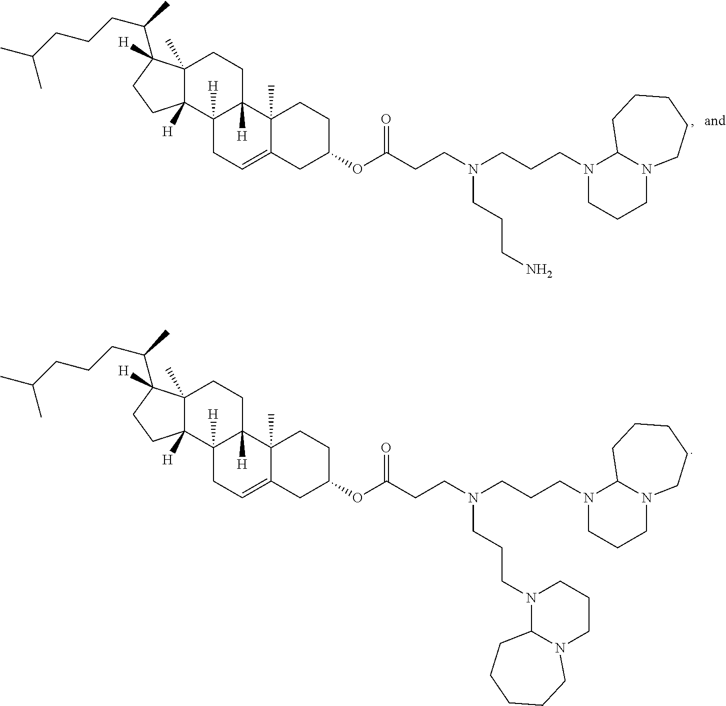

More preferably, the nanoparticle composition includes the cationic lipid having the structure:

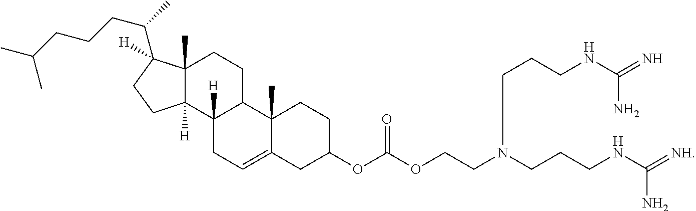

In a further aspect of the invention, the nanoparticle composition described herein can include additional cationic lipids. Additional suitable lipids contemplated include, for example:

N-(1-(2,3-dioleoyloxy)propyl)-N,N,N-trimethylammonium chloride (DOTMA);

1,2-dioleoyloxy-3-(trimethylammonium)propane or N-(2,3-dioleoyloxy)propyl)-N,N,N-trimethylammonium chloride (DOTAP);

1,2-dimyrstoyloxy-3-(trimethylammonia)propane (DMTAP);

1,2-dimyristyloxypropyl-3-dimethylhydroxyethylammonium bromide or N-(1,2-dimyristyloxyprop-3-yl)-N,N-dimethyl-N-hydroxyethyl ammonium bromide (DMRIE);

dimethyldidodecylammonium bromide (DDAB);

3-(N-(N′,N′-dimethylaminoethane)carbamoyl)cholesterol (DC-Cholesterol);

3β-((N′,N′-diguanidinoethyl-aminoethane)carbamoyl)cholesterol (BGTC);

2-(2-(3-(bis(3-aminopropyl)amino)propylamino)acetamido)-N,N-ditetradecylacetamide (RPR209120);

1,2-dialkenoyl-sn-glycero-3-ethylphosphocholines (i.e., 1,2-dioleoyl-sn-glycero-3-ethylphosphocholine, 1,2-distearoyl-sn-glycero-3-ethylphosphocholine and 1,2-dipalmitoyl-sn-glycero-3-ethylphosphocholine);

tetramethyltetrapalmitoyl spermine (TMTPS);

tetramethyltetraoleyl spermine (TMTOS);

tetramethyltetralauryl spermine (TMTLS);

tetramethyltetramyristyl spermine (TMTMS);

tetramethyldioleyl spermine (TMDOS);

2,5-bis(3-aminopropylamino)-N-(2-(dioctadecylamino)-2-oxoethyl)pentanamide (DOGS);

2,5-bis(3-aminopropylamino)-N-(2-(di(Z)-octadeca-9-dienylamino)-2-oxoethyl)pentanamide (DOGS-9-en);

2,5-bis(3-aminopropylamino)-N-(2-(di(9Z,12Z)-octadeca-9,12-dienylamino)-2-oxoethyl)pentanamide (DLinGS);

N4-Spermine cholesteryl carbamate (GL-67);

(9Z,9′Z)-2-(2,5-bis(3-aminopropylamino)pentanamido)propane-1,3-diyl-dioctadec-9-enoate (DOSPER);

2,3-dioleyloxy-N-[2(sperminecarboxamido)ethyl]-N,N-dimethyl-1-propanaminium trifluoroacetate (DOSPA);

1,2-dimyristoyl-3-trimethylammonium-propane; 1,2-distearoyl-3-trimethylammonium-propane;

dioctadecyldimethylammonium (DODMA);

dimethyldioctadecylammonium (DODAB);

distearyldimethylammonium (DSDMA);

N,N-dioleyl-N,N-dimethylammonium chloride (DODAC); and pharmaceutically acceptable salts thereof and mixtures thereof.

Details of cationic lipids are also described in US2007/0293449 and U.S. Pat. Nos. 4,897,355; 5,279,833; 6,733,777; 6,376,248; 5,736,392; 5,686,958; 5,334,761; 5,459,127; 2005/0064595; U.S. Pat. Nos. 5,208,036; 5,264,618; 5,279,833; 5,283,185; 5,753,613; and 5,785,992.

Additionally, commercially available preparations including cationic lipids can be used: for example, LIPOFECTIN® (cationic liposomes containing DOTMA and DOPE, from GIBCO/BRL, Grand Island, N.Y., USA); LIPOFECTAMINE® (cationic liposomes containing DOSPA and DOPE, from GIBCO/BRL, Grand Island, N.Y., USA); and TRANSFECTAM® (cationic liposomes containing DOGS from Promega Corp., Madison, Wis., USA).

### C. Fusogenic/Non-Cationic Lipids

In another aspect of the invention, the nanoparticle composition contains a fusogenic lipid. The fusogenic lipids include non-cationic lipids such as neutral uncharged, zwitter ionic and anionic lipids. For purposes of the present invention, the terms “fusogenic lipid” and “non-cationic lipids” are interchangeable.

Neutral lipids include a lipid that exists either in an uncharged or neutral zwitter ionic form at a selected pH, preferably at physiological pH. Examples of such lipids include diacylphosphatidylcholine, diacylphosphatidylethanolamine, ceramide, sphingomyelin, cephalin, cholesterol, cerebrosides and diacylglycerols.

Anionic lipids include a lipid that is negatively charged at physiological pH. These lipids include, but are not limited to, phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and neutral lipids modified with other anionic modifying groups.

Many fusogenic lipids include amphipathic lipids generally having a hydrophobic moiety and a polar head group, and can form vesicles in aqueous solution.

Fusogenic lipids contemplated include naturally-occurring and synthetic phospholipids and related lipids.

A non-limiting list of the non-cationic lipids are selected from among phospholipids and nonphosphous lipid-based materials, such as lecithin; lysolecithin; diacylphosphatidylcholine; lysophosphatidylcholine; phosphatidylethanolamine; lysophosphatidylethanolamine; phosphatidylserine; phosphatidylinositol; sphingomyelin; cephalin; ceramide; cardiolipin; phosphatidic acid; phosphatidylglycerol; cerebrosides; dicetylphosphate;

1,2-dilauroyl-sn-glycerol (DLG);

1,2-dimyristoyl-sn-glycerol (DMG);

1,2-dipalmitoyl-sn-glycerol (DPG);

1,2-distearoyl-sn-glycerol (DSG);

1,2-dilauroyl-sn-glycero-3-phosphatidic acid (DLPA);

1,2-dimyristoyl-sn-glycero-3-phosphatidic acid (DMPA);

1,2-dipalmitoyl-sn-glycero-3-phosphatidic acid (DPPA);

1,2-distearoyl-sn-glycero-3-phosphatidic acid (DSPA);

1,2-diarachidoyl-sn-glycero-3-phosphocholine (DAPC);

1,2-dilauroyl-sn-glycero-3-phosphocholine (DLPC);

1,2-dimyristoyl-sn-glycero-3-phosphocholine (DMPC);

1,2-dipalmitoyl-sn-glycero-3-ethylphosphocholine (DPePC);

1,2-dipalmitoyl-sn-glycero-3-phosphocholine or dipalmitoylphosphatidylcholine (DPPC);

1,2-distearoyl-sn-glycero-3-phosphocholine or distearoylphosphatidylcholine (DSPC);

1,2-dilauroyl-sn-glycero-3-phosphoethanolamine (DLPE);

1,2-dimyristoyl-sn-glycero-3-phosphoethanolamine or dimyristoylphosphoethanolamine (DMPE);

1,2-dipalmitoyl-sn-glycero-3-phosphoethanolamine or dipalmitoylphosphatidylethanolamine (DPPE);

1,2-distearoyl-sn-glycero-3-phosphoethanolamine or distearoylphosphatidylethanolamine (DSPE);

1,2-dioleoyl-sn-glycero-3-phosphoethanolamine or dioleoylphosphatidylethanolamine (DOPE);

1,2-dilauroyl-sn-glycero-3-phosphoglycerol (DLPG);

1,2-dimyristoyl-sn-glycero-3-phosphoglycerol (DMPG) or 1,2-dimyristoyl-sn-glycero-3-phospho-sn-1-glycerol (DMP-sn-1-G);

1,2-dipalmitoyl-sn-glycero-3-phosphoglycerol or dipalmitoylphosphatidylglycerol (DPPG);

1,2-distearoyl-sn-glycero-3-phosphoglycerol (DSPG) or 1,2-distearoyl-sn-glycero-3-phospho-sn-1-glycerol (DSP-sn-1-G);

1,2-dipalmitoyl-sn-glycero-3-phospho-L-serine (DPPS);

1-palmitoyl-2-linoleoyl-sn-glycero-3-phosphocholine (PLinoPC);

1-palmitoyl-2-oleoyl-sn-glycero-3-phosphocholine or palmitoyloleoylphosphatidylcholine (POPC);

1-palmitoyl-2-oleoyl-sn-glycero-3-phosphoglycerol (POPG);

1-palmitoyl-2-lyso-sn-glycero-3-phosphocholine (P-lyso-PC);

1-stearoyl-2-lyso-sn-glycero-3-phosphocholine (S-lyso-PC);

diphytanoylphosphatidylethanolamine (DPhPE);

1,2-dioleoyl-sn-glycero-3-phosphocholine or dioleoylphosphatidylcholine (DOPC);

1,2-diphytanoyl-sn-glycero-3-phosphocholine (DPhPC),

dioleoylphosphatidylglycerol (DOPG);

palmitoyloleoylphosphatidylethanolamine (POPE);

dioleoyl-phosphatidylethanolamine 4-(N-maleimidomethyl)-cyclohexane-1-carboxylate (DOPE-mal);

16-O-monomethyl PE;

16-O-dimethyl PE;

18-1-trans PE; 1-stearoyl-2-oleoyl-phosphatidylethanolamine (SOPE);

1,2-dielaidoyl-sn-glycero-3-phosphoethanolamine (transDOPE); and pharmaceutically acceptable salts thereof and mixtures thereof. Details of the fusogenic lipids are described in US Patent Publication Nos. 2007/0293449 and 2006/0051405.

Noncationic lipids include sterols or steroid alcohols such as cholesterol.

Additional non-cationic lipids are, e.g., stearylamine, dodecylamine, hexadecylamine, acetylpalmitate, glycerolricinoleate, hexadecylstereate, isopropylmyristate, amphoteric acrylic polymers, triethanolaminelauryl sulfate, alkylarylsulfate polyethyloxylated fatty acid amides, and dioctadecyldimethyl ammonium bromide.

Anionic lipids contemplated include phosphatidylserine, phosphatidic acid, phosphatidylcholine, platelet-activation factor (PAF), phosphatidylethanolamine, phosphatidyl-DL-glycerol, phosphatidylinositol, phosphatidylinositol, cardiolipin, lysophosphatides, hydrogenated phospholipids, sphingoplipids, gangliosides, phytosphingosine, sphinganines, pharmaceutically acceptable salts and mixtures thereof.

Suitable noncationic lipids useful for the preparation of the nanoparticle composition described herein include diacylphosphatidylcholine (e.g., distearoylphosphatidylcholine, dioleoylphosphatidylcholine, dipalmitoylphosphatidylcholine and dilinoleoylphosphatidylcholine), diacylphosphatidylethanolamine (e.g., dioleoylphosphatidylethanolamine and palmitoyloleoylphosphatidylethanolamine), ceramide or sphingomyelin. The acyl groups in these lipids are preferably fatty acids having saturated and unsaturated carbon chains such as linoyl, isostearyl, oleyl, elaidyl, petroselinyl, linolenyl, elaeostearyl, arachidyl, myristoyl, palmitoyl, and lauroyl. More preferably, the acyl groups are lauroyl, myristoyl, palmitoyl, stearoyl or oleoyl. Alternatively and/preferably, the fatty acids have saturated and unsaturated C₈-C₃₀ (preferably C₁₀-C₂₄) carbon chains.

A variety of phosphatidylcholines useful in the nanoparticle composition described herein includes:

1,2-didecanoyl-sn-glycero-3-phosphocholine (DDPC, C10:0, C10:0);

1,2-dilauroyl-sn-glycero-3-phosphocholine (DLPC, C12:0, C12:0);

1,2-dimyristoyl-sn-glycero-3-phosphocholine (DMPC, C14:0, C14:0);

1,2-dipalmitoyl-sn-glycero-3-phosphocholine (DPPC, C16:0, C16:0);

1,2-distearoyl-sn-glycero-3-phosphocholine (DSPC, C18:0, C18:0);

1,2-dioleoyl-sn-glycero-3-phosphocholine (DOPC, C18:1, C18:1);

1,2-dierucoyl-sn-glycero-3-phosphocholine (DEPC, C22:1, C22:1);

1,2-dieicosapentaenoyl-sn-glycero-3-phosphocholine (EPA-PC, C20:5, C20:5);

1,2-didocosahexaenoyl-sn-glycero-3-phosphocholine (DHA-PC, C22:6, C22:6);

1-myristoyl-2-palmitoyl-sn-glycero-3-phosphocholine (MPPC, C14:0, C16:0);

1-myristoyl-2-stearoyl-sn-glycero-3-phosphocholine (MSPC, C14:0, C18:0);

1-palmitoyl-2-stearoyl-sn-glycero-3-phosphocholine (PMPC, C16:0, C14:0);

1-palmitoyl-2-stearoyl-sn-glycero-3-phosphocholine (PSPC, C16:0, C18:0);

1-stearoyl-2-myristoyl-sn-glycero-3-phosphocholine (SMPC, C18:0, C14:0);

1-stearoyl-2-palmitoyl-sn-glycero-3-phosphocholine (SPPC, C18:0, C16:0);

1,2-myristoyl-oleoyl-sn-glycero-3-phosphoethanolamine (MOPC, C14:0, C18:0);

1,2-palmitoyl-oleoyl-sn-glycero-3-phosphoethanolamine (POPC, C16:0, C18:1);

1,2-stearoyl-oleoyl-sn-glycero-3-phosphoethanolamine (POPC, C18:0, C18:1), and pharmaceutically acceptable salts thereof and mixtures thereof.

A variety of lysophosphatidylcholine useful in the nanoparticle composition described herein includes:

1-myristoyl-2-lyso-sn-glycero-3-phosphocholine (M-LysoPC, C14:0);

1-malmitoyl-2-lyso-sn-glycero-3-phosphocholine (P-LysoPC, C16:0);

1-stearoyl-2-lyso-sn-glycero-3-phosphocholine (S-LysoPC, C18:0), and pharmaceutically acceptable salts thereof and mixtures thereof.

A variety of phosphatidylglycerols useful in the nanoparticle composition described herein are selected from among:

hydrogenated soybean phosphatidylglycerol (HSPG);

non-hydrogenated egg phosphatidylglycerol (EPG);

1,2-dimyristoyl-sn-glycero-3-phosphoglycerol (DMPG, C14:0, C14:0);

1,2-dipalmitoyl-sn-glycero-3-phosphoglycerol (DPPG, C16:0, C16:0);

1,2-distearoyl-sn-glycero-3-phosphoglycerol (DSPG, C18:0, C18:0);

1,2-dioleoyl-sn-glycero-3-phosphoglycerol (DOPG, C18:1, C18:1);

1,2-dierucoyl-sn-glycero-3-phosphoglycerol (DEPG, C22:1, C22:1);

1-palmitoyl-2-oleoyl-sn-glycero-3-phosphoglycerol (POPG, C16:0, C18:1), and pharmaceutically acceptable salts thereof and mixtures thereof.

A variety of phosphatidic acids useful in the nanoparticle composition described herein includes:

1,2-dimyristoyl-sn-glycero-3-phosphatidic acid (DMPA, C14:0, C14:0);

1,2-dipalmitoyl-sn-glycero-3-phosphatidic acid (DPPA, C16:0, C16:0);

1,2-distearoyl-sn-glycero-3-phosphatidic acid (DSPA, C18:0, C18:0), and pharmaceutically acceptable salts thereof and mixtures thereof.

A variety of phosphatidylethanolamines useful in the nanoparticle composition described herein includes:

hydrogenated soybean phosphatidylethanolamine (HSPE);

non-hydrogenated egg phosphatidylethanolamine (EPE);

1,2-dimyristoyl-sn-glycero-3-phosphoethanolamine (DMPE, C14:0, C14:0);

1,2-dipalmitoyl-sn-glycero-3-phosphoethanolamine (DPPE, C16:0, C16:0);

1,2-distearoyl-sn-glycero-3-phosphoethanolamine (DSPE, C18:0, C18:0);

1,2-dioleoyl-sn-glycero-3-phosphoethanolamine (DOPE, C18:1, C18:1);

1,2-dioleoyl-sn-glycero-3-phosphoethanolamine (DEPE, C22:1, C22:1);

1,2-dierucoyl-sn-glycero-3-phosphoethanolamine (POPE, C16:0, C18:1), and pharmaceutically acceptable salts thereof and mixtures thereof.

A variety of phosphatidylserines useful in the nanoparticle composition described herein includes:

1,2-dimyristoyl-sn-glycero-3-phospho-L-serine (DMPS, C14:0, C14:0);

1,2-dipalmitoyl-sn-glycero-3-phospho-L-serine (DPPS, C16:0, C16:0);

1,2-distearoyl-sn-glycero-3-phospho-L-serine (DSPS, C18:0, C18:0);

1,2-dioleoyl-sn-glycero-3-phospho-L-serine (DOPS, C18:1, C18:1);

1-palmitoyl-2-oleoyl-sn-3-phospho-L-serine (POPS, C16:0, C18:1), and pharmaceutically acceptable salts thereof and mixtures thereof.

In one preferred embodiment, suitable neutral lipids useful for the preparation of the nanoparticle composition described herein include, for example,

dioleoylphosphatidylethanolamine (DOPE),

distearoylphosphatidylethanolamine (DSPE),

palmitoyloleoylphosphatidylethanolamine (POPE),

egg phosphatidylcholine (EPC),

dipalmitoylphosphatidylcholine (DPPC),

distearoylphosphatidylcholine (DSPC),

dioleoylphosphatidylcholine (DOPC),

palmitoyloleoylphosphatidylcholine (POPC),

dipalmitoylphosphatidylglycerol (DPPG),

dioleoylphosphatidylglycerol (DOPG),

dioleoyl-phosphatidylethanolamine 4-(N-maleimidomethyl)-cyclohexane-1-carboxylate (DOPE-mal), cholesterol, pharmaceutically acceptable salts and mixtures thereof.

In certain preferred embodiments, the nanoparticle composition described herein includes DSPC, EPC, DOPE, etc, and mixtures thereof.

In a further aspect of the invention, the nanoparticle composition contains non-cationic lipids such as sterol. The nanoparticle composition preferably contains cholesterol or analogs thereof, and more preferably cholesterol.

### D. PEG Lipids

In another aspect of the invention, the nanoparticle composition described herein contains a PEG lipid. The PEG lipids extend circulation of the nanoparticle described herein and prevent the premature excretion of the nanoparticles from the body. The PEG lipids allow a reduction in the immune response in the body. The PEG lipids also enhance stability of the nanoparticles.

The PEG lipids useful in the nanoparticle composition include PEGylated forms of fusogenic/noncationic lipids. The PEG lipids include, for example, PEG conjugated to diacylglycerols (PEG-DAG), PEG conjugated to diacylglycamides, PEG conjugated to dialkyloxypropyls (PEG-DAA), PEG conjugated to phospholipids such as PEG coupled to phosphatidylethanolamine (PEG-PE), PEG conjugated to ceramides (PEG-Cer), PEG conjugated to cholesterol derivatives (PEG-Chol) or mixtures thereof. See U.S. Pat. Nos. 5,885,613 and 5,820,873, and US Patent Publication No. 2006/051405, the contents of each of which are incorporated herein by reference.

PEG is generally represented by the structure:

—O—(CH₂CH₂O)ₙ—

where (n) is a positive integer from about 5 to about 2300, preferably from about 5 to about 460 so that the polymeric portion of PEG lipid has an average number molecular weight of from about 200 to about 100,000 daltons, preferably from about 200 to about 20,000 daltons.

Alternatively, the polyethylene glycol (PEG) residue portion can be represented by the structure:

—Y₇₁—(CH₂CH₂O)ₙ—CH₂CH₂Y₇₁—,

—Y₇₁—(CH₂CH₂O)ₙ—CH₂C(═Y₇₂)—Y₇₁—,

—Y₇₁—C(═Y₇₂)—(CH₂)ₐ₂—Y₇₃—(CH₂CH₂O)ₙ—CH₂CH₂—Y₇₃—(CH₂)ₐ₂—C(═Y₇₂)—Y₇₁— and

—Y₇₁—(CR₇₁R₇₂)ₐ₂—Y₇₃—(CH₂)_{b2}—O—(CH₂CH₂O)ₙ—(CH₂)_{b2}—Y₇₃—(CR₇₁R₇₂)ₐ₂—Y₇₁—,

wherein:

Y₇₁ and Y₇₃ are independently O, S, SO, SO₂, NR₇₃ or a bond;

Y₇₂ is O, S, or NR₇₄;

R₇₁₋₇₄ are independently selected from among hydrogen, C₁₋₆ alkyl, C₂₋₆ alkenyl, C₂₋₆ alkynyl, C₃₋₁₉ branched alkyl, C₃₋₈ cycloalkyl, C₁₋₆ substituted alkyl, C₂₋₆ substituted alkenyl, C₂₋₆ substituted alkynyl, C₃₋₈ substituted cycloalkyl, aryl, substituted aryl, heteroaryl, substituted heteroaryl, C₁₋₆ heteroalkyl, substituted C₁₋₆ heteroalkyl, C₁₋₆ alkoxy, aryloxy, C₁₋₆ heteroalkoxy, heteroaryloxy, C₂₋₆ alkanoyl, arylcarbonyl, C₂₋₆ alkoxycarbonyl, aryloxycarbonyl, C₂₋₆ alkanoyloxy, arylcarbonyloxy, C₂₋₆ substituted alkanoyl, substituted arylcarbonyl, C₂₋₆ substituted alkanoyloxy, substituted aryloxycarbonyl, C₂₋₆ substituted alkanoyloxy and substituted arylcarbonyloxy, preferably hydrogen, methyl, ethyl or propyl;

(a2) and (b2) are independently zero or a positive integer, preferably zero or an integer from about 1 to about 6 (i.e., 1, 2, 3, 4, 5, 6), and more preferably 1 or 2; and

(n) is an integer from about 5 to about 2300, preferably from about 5 to about 460.

The terminal end of PEG can end with H, NH₂, OH, CO₂H, C₁₋₆ alkyl (e.g., methyl, ethyl, propyl), C₁₋₆ alkoxy, acyl or aryl. In a preferred embodiment, the terminal hydroxyl group of PEG is substituted with a methoxy or methyl group. In one preferred embodiment, the PEG employed in the PEG lipid is methoxy PEG.

The PEG may be directly conjugated to lipids or via a linker moiety. The polymers for conjugation to a lipid structure are converted into a suitably activated polymer, using the activation techniques described in U.S. Pat. Nos. 5,122,614 and 5,808,096 and other techniques known in the art without undue experimentation.

Examples of activated PEGs useful for the preparation of a PEG lipid include, for example, methoxypolyethylene glycol-succinate, mPEG-NHS, methoxypolyethylene glycol-succinimidyl succinate, methoxypolyethyleneglycol-acetic acid (mPEG-CH₂COOH), methoxypolyethylene glycol-amine (mPEG-NH₂), and methoxypolyethylene glycol-tresylate (mPEG-TRES).

In certain aspects, polymers having terminal carboxylic acid groups can be employed in the PEG lipids described herein. Methods of preparing polymers having terminal carboxylic acids in high purity are described in U.S. patent application Ser. No. 11/328,662, the contents of which are incorporated herein by reference.

In alternative aspects, polymers having terminal amine groups can be employed to make the PEG-lipids described herein. The methods of preparing polymers containing terminal amines in high purity are described in U.S. patent application Ser. Nos. 11/508,507 and 11/537,172, the contents of each of which are incorporated by reference.

PEG and lipids can be bound via a linkage, i.e. a non-ester containing linker moiety or an ester containing linker moiety. Suitable non-ester containing linkers include, but are not limited to, an amido linker moiety, an amino linker moiety, a carbonyl linker moiety, a carbamate linker moiety, a carbonate (OC(═O)O) linker moiety, a urea linker moiety, an ether linker moiety, a succinyl linker moiety, and combinations thereof. Suitable ester linker moieties include, e.g., succinoyl, phosphate esters (—O—P(═O)(OH)—O—), sulfonate esters, and combinations thereof.

In one embodiment, the nanoparticle composition described herein includes a polyethyleneglycol-diacylglycerol (PEG-DAG) or polyethylene-diacylglycamide. Suitable polyethyleneglycol-diacylglycerol or polyethyleneglycol-diacylglycamide conjugates include a dialkylglycerol or dialkylglycamide group having alkyl chain length independently containing from about C₄ to about C₃₀ (preferably from about C₈ to about C₂₄) saturated or unsaturated carbon atoms. The dialkylglycerol or dialkylglycamide group can further include one or more substituted alkyl groups.

The term “diacylglycerol” (DAG) used herein refers to a compound having two fatty acyl chains, R₁₁ and R₁₂. DAG has the general formula:

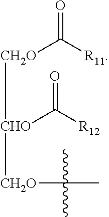

The R₁₁ and R₁₂ have the same or different about 4 to about 30 carbons (preferably about 8 to about 24) and are bonded to glycerol by ester linkages. The acyl groups can be saturated or unsaturated with various degrees of unsaturation.

In a preferred embodiment, the PEG-diacylglycerol conjugate is a PEG-dilaurylglycerol (C12), a PEG-dimyristylglycerol (C14, DMG), a PEG-dipalmitoylglycerol (C16, DPG) or a PEG-distearylglycerol (C18, DSG). Those of skill in the art will readily appreciate that other diacylglycerols are also contemplated in the PEG-diacylglycol conjugate. Suitable PEG-diacylglycerol conjugates for use in the present invention, and methods of making and using them, are described in U.S. Patent Publication No. 2003/0077829, and PCT Patent Application No. CA 02/00669, the contents of each of which are incorporated herein by reference.

Examples of the PEG-diacylglycerol conjugate can be selected from among PEG-dilaurylglycerol (C12), PEG-dimyristylglycerol (C14), PEG-dipalmitoylglycerol (C16), PEG-disterylglycerol (C18). Examples of the PEG-diacylglycamide conjugate include PEG-dilaurylglycamide (C12), PEG-dimyristylglycamide (C14), PEG-dipalmitoyl-glycamide (C16), and PEG-disterylglycamide (C18).

In another embodiment, the nanoparticle composition described herein includes a polyethyleneglycol-dialkyloxypropyl conjugates (PEG-DAA).

The term “dialkyloxypropyl” refers to a compound having two alkyl chains, R₁₁ and R₁₂. The R₁₁ and R₁₂ alkyl groups include the same or different between about 4 to about 30 carbons (preferably about 8 to about 24). The alkyl groups can be saturated or have varying degrees of unsaturation. Dialkyloxypropyls have the general formula:

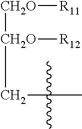

wherein R₁₁ and R₁₂ alkyl groups are the same or different alkyl groups having from about 4 to about 30 carbons (preferably about 8 to about 24). The alkyl groups can be saturated or unsaturated. Suitable alkyl groups include, but are not limited to, lauryl (C12), myristyl (C14), palmityl (C16), stearyl (C18), oleoyl (C18) and icosyl (C20).

In one embodiment, R₁₁ and R₁₂ are both the same, i.e., R₁₁ and R₁₂ are both myristyl (C14) or both stearyl (C18), or both oleoyl (C18), etc. In another embodiment, R₁₁ and R₁₂ are different, i.e., R₁₁ is myristyl (C14) and R₁₂ is stearyl (C18). In a preferred embodiment, the PEG-dialkylpropyl conjugates include the same R₁₁ and R₁₂.

In yet another embodiment, the nanoparticle composition described herein includes PEG conjugated to phosphatidylethanolamines (PEG-PE). The phosphatidylethanolamines useful for the PEG lipid conjugation can contain saturated or unsaturated fatty acids with carbon chain lengths in the range of about 4 to about 30 carbons (preferably about 8 to about 24). Suitable phosphatidylethanolamines include, but are not limited to: dimyristoylphosphatidylethanolamine (DMPE), dipalmitoylphosphatidylethanolamine (DPPE), dioleoylphosphatidylethanolamine (DOPE) and distearoylphosphatidylethanolamine (DSPE).

In yet another embodiment, the nanoparticle composition described herein includes PEG conjugated to ceramides (PEG-Cer). Ceramides have only one acyl group. Ceramides can have saturated or unsaturated fatty acids with carbon chain lengths in the range of about 4 to about 30 carbons (preferably about 8 to about 24).

In alternative embodiments, the nanoparticle composition described herein includes PEG conjugated to cholesterol derivatives. The term “cholesterol derivative” means any cholesterol analog containing a cholesterol structure with modification, i.e., substitutions and/or deletions thereof. The term cholesterol derivative herein also includes steroid hormones and bile acids.

In one preferred aspect, the PEG is a polyethylene glycol with an average number molecular weight ranging from about 200 to about 20,000 daltons, more preferably from about 500 to about 10,000 daltons, yet more preferably about 1,000 to about 5,000 daltons (i.e., about 1,500 to about 3,000 daltons). In one particular embodiment, the PEG has an average number molecular weight of about 2,000 daltons. In another particular embodiment, the PEG has an average number molecular weight of about 750 daltons.

Illustrative examples of PEG lipids include N-(carbonyl-methoxypolyethyleneglycol)-1,2-dimyristoyl-sn-glycero-3-phosphoethanolamine (^{2 kDa}mPEG-DMPE or ^{5 kDa}mPEG-DMPE); N-(carbonyl-methoxypolyethyleneglycol)-1,2-dipalmitoyl-sn-glycero-3-phosphoethanolamine (^{2 kDa}mPEG-DPPE or ^{5 kDa}mPEG-DPPE); N-(carbonyl-methoxypolyethyleneglycol)-1,2-distearoyl-sn-glycero-3-phosphoethanolamine (^{750 Da}mPEG-DSPE, ^{2 kDa}mPEG-DSPE, ^{5 kDa}mPEG-DSPE); and pharmaceutically acceptable salts (i.e., sodium salt) thereof and mixtures thereof.

In certain preferred embodiments, the nanoparticle composition described herein includes a PEG lipid having PEG-DAG or PEG-ceramide, wherein PEG has molecular weight from about 200 to about 20,000, preferably from about 500 to about 10,000, and more preferably from about 1,000 to about 5,000.

A few illustrative embodiments of PEG-DAG and PEG-ceramide are provided in Table 1.

TABLE 1
PEG-Lipid

| | |
|---|---|
| PEG-DAG | mPEG-diimyristoylglycerol |
| | mPEG-dipalmitoylglycerol |
| | mPEG-distearoylglycerol |
| PEG-Ceramide | mPEG-CerC8 |
| | mPEG-CerC14 |
| | mPEG-CerC16 |
| | mPEG-CerC20 |

Preferably, the nanoparticle composition described herein includes the PEG lipid selected from among PEG-DSPE, PEG-dipalmitoylglycamide (C16), PEG-Ceramide (C16), etc. and mixtures thereof. The structures of mPEG-DSPE, mPEG-dipalmitoylglycamide (C16), and mPEG-Ceramide (C16) are as follows:

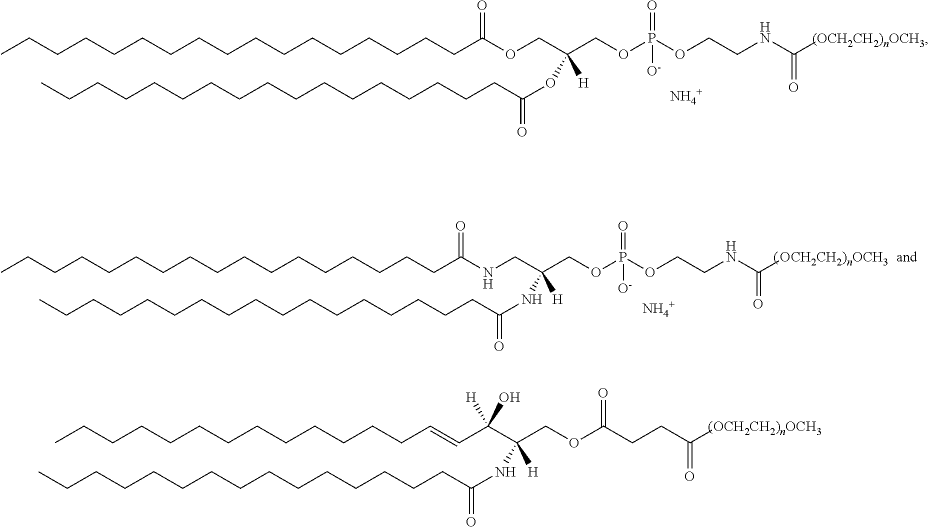

wherein, (n) is an integer from about 5 to about 2300, preferably from about 5 to about 460.

In one particular embodiment, (n) is about 45.

In a further embodiment and as an alternative to PAO-based polymers such as PEG, one or more effectively non-antigenic materials such as dextran, polyvinyl alcohols, carbohydrate-based polymers, hydroxypropylmethacrylamide (HPMA), polyalkylene oxides, and/or copolymers thereof can be used. Examples of suitable polymers that can be used in place of PEG include, but are not limited to, polyvinylpyrrolidone, polymethyloxazoline, polyethyloxazoline, polyhydroxypropyl methacrylamide, polymethacrylamide and polydimethylacrylamide, polylactic acid, polyglycolic acid, and derivatized celluloses, such as hydroxymethylcellulose or hydroxyethylcellulose. See also commonly-assigned U.S. Pat. No. 6,153,655, the contents of which are incorporated herein by reference. It will be understood by those of ordinary skill that the same type of activation can be employed as described herein as for PAOs such as PEG. Those of ordinary skill in the art will further realize that the foregoing list is merely illustrative and that all polymeric materials having the qualities described herein are contemplated. For purposes of the present invention, “substantially or effectively non-antigenic” means all materials understood in the art as being nontoxic and not eliciting an appreciable immunogenic response in mammals.

### E. Nucleic Acids/Oligonucleotides

The nanoparticle compositions described herein can be used for delivering various nucleic acids into cells or tissues. The nucleic acids include plasmids and oligonucleotides. Preferably, the nanoparticle compositions described herein are used for delivery of oligonucleotides.

In order to more fully appreciate the scope of the present invention, the following terms are defined. The artisan will appreciate that the terms, “nucleic acid” or “nucleotide” apply to deoxyribonucleic acid (“DNA”), ribonucleic acid, (“RNA”) whether single-stranded or double-stranded, unless otherwise specified, and any chemical modifications thereof. An “oligonucleotide” is generally a relatively short polynucleotide, e.g., ranging in size from about 2 to about 200 nucleotides, preferably from about 8 to about 50 nucleotides, more preferably from about 8 to about 30 nucleotides, and yet more preferably from about 8 to about 20 or from about 15 to about 28 in length. The oligonucleotides according to the invention are generally synthetic nucleic acids, and are single stranded, unless otherwise specified. The terms, “polynucleotide” and “polynucleic acid” may also be used synonymously herein.

The oligonucleotides (analogs) are not limited to a single species of oligonucleotide but, instead, are designed to work with a wide variety of such moieties, it being understood that linkers can attach to one or more of the 3′- or 5′-terminals, usually PO₄ or SO₄ groups of a nucleotide. The nucleic acid molecules contemplated can include a phosphorothioate internucleotide linkage modification, sugar modification, nucleic acid base modification and/or phosphate backbone modification. The oligonucleotides can contain natural a phosphorodiester backbone or phosphorothioate backbone or any other modified backbone analogues such as LNA (Locked Nucleic Acid), PNA (nucleic acid with peptide backbone), CpG oligomers, and the like, such as those disclosed at Tides 2002, Oligonucleotide and Peptide Technology Conferences, May 6-8, 2002, Las Vegas, Nev. and Oligonucleotide & Peptide Technologies, 18th & 19th Nov. 2003, Hamburg, Germany, the contents of which are incorporated herein by reference.

Modifications to the oligonucleotides contemplated by the invention include, for example, the addition or substitution of functional moieties that incorporate additional charge, polarizability, hydrogen bonding, electrostatic interaction, and functionality to an oligonucleotide. Such modifications include, but are not limited to, 2′-position sugar modifications, 5-position pyrimidine modifications, 8-position purine modifications, modifications at exocyclic amines, substitution of 4-thiouridine, substitution of 5-bromo or 5-iodouracil, backbone modifications, methylations, base-pairing combinations such as the isobases isocytidine and isoguanidine, and analogous combinations. Oligonucleotides contemplated within the scope of the present invention can also include 3′ and/or 5′ cap structure

For purposes of the present invention, “cap structure” shall be understood to mean chemical modifications, which have been incorporated at either terminus of the oligonucleotide. The cap can be present at the 5′-terminus (5′-cap) or at the 3′-terminus (3′-cap) or can be present on both termini. A non-limiting example of the 5′-cap includes inverted abasic residue (moiety), 4′,5′-methylene nucleotide; 1-(beta-D-erythrofuranosyl) nucleotide, 4′-thio nucleotide, carbocyclic nucleotide; 1,5-anhydrohexitol nucleotide; L-nucleotides; alpha-nucleotides; modified base nucleotide; phosphorodithioate linkage; threo-pentofuranosyl nucleotide; acyclic 3′,4′-seco nucleotide; acyclic 3,4-dihydroxybutyl nucleotide; acyclic 3,5-dihydroxypentyl nucleotide; 3′-3′-inverted nucleotide moiety; 3′-3′-inverted abasic moiety; 3′-2′-inverted nucleotide moiety; 3′-2′-inverted abasic moiety; 1,4-butanediol phosphate; 3′-phosphoramidate; hexylphosphate; aminohexyl phosphate; 3′-phosphate; 3′-phosphorothioate; phosphorodithioate; or bridging or non-bridging methylphosphonate moiety. Details are described in WO 97/26270, incorporated by reference herein. The 3′-cap can include, for example, 4′,5′-methylene nucleotide; 1-(beta-D-erythrofuranosyl)nucleotide; 4′-thio nucleotide, carbocyclic nucleotide; 5′-aminoalkyl phosphate; 1,3-diamino-2-propyl phosphate; 3-aminopropyl phosphate; 6-aminohexyl phosphate; 1,2-aminododecyl phosphate; hydroxypropyl phosphate; 1,5-anhydrohexitol nucleotide; L-nucleotide; alpha-nucleotide; modified base nucleotide; phosphorodithioate; threopentofuranosyl nucleotide; acyclic 3′,4′-seco nucleotide; 3,4-dihydroxybutyl nucleotide; 3,5-dihydroxypentyl nucleotide, 5′-5′-inverted nucleotide moiety; 5′-5′-inverted abasic moiety; 5′-phosphoramidate; 5′-phosphorothioate; 1,4-butanediol phosphate; 5′-amino; bridging and/or non-bridging 5′-phosphoramidate, phosphorothioate and/or phosphorodithioate, bridging or non bridging methylphosphonate and 5′-mercapto moieties. See also Beaucage and Iyer, 1993, Tetrahedron 49, 1925; the contents of which are incorporated by reference herein.

A non-limiting list of nucleoside analogs have the structure:

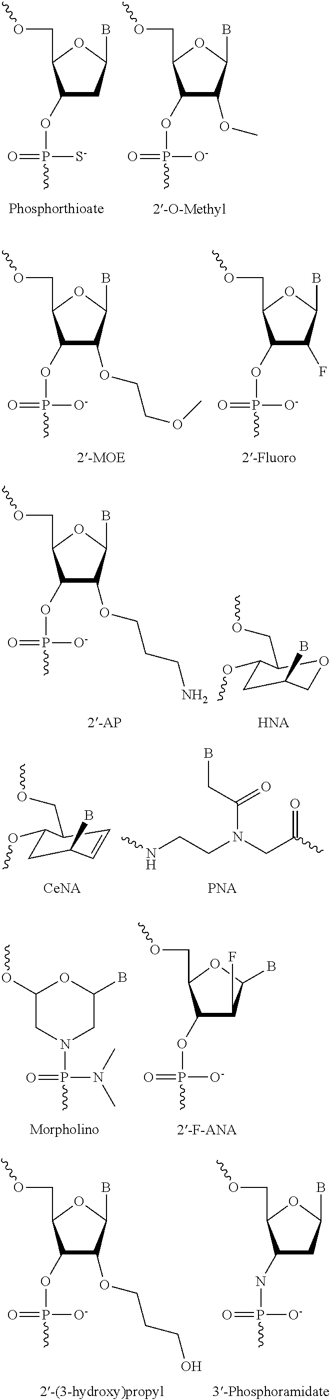

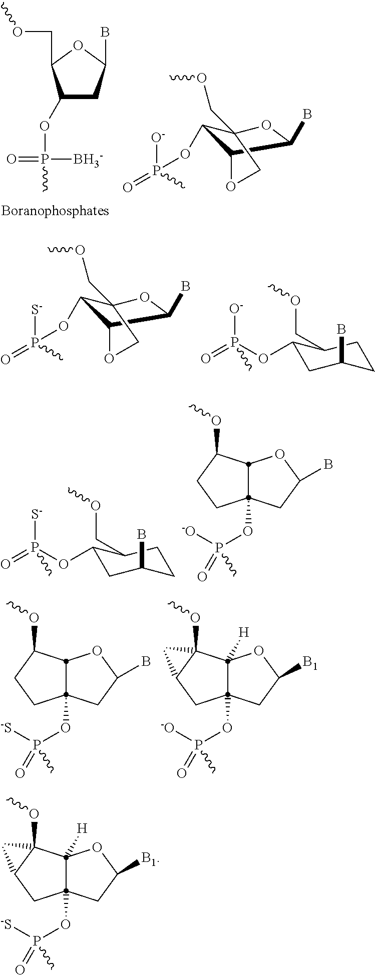

See more examples of nucleoside analogues described in Freier & Altmann; Nucl. Acid Res., 1997, 25, 4429-4443 and Uhlmann; Curr. Opinion in Drug Development, 2000, 3(2), 293-213, the contents of each of which are incorporated herein by reference.

The term “antisense,” as used herein, refers to nucleotide sequences which are complementary to a specific DNA or RNA sequence that encodes a gene product or that encodes a control sequence. The term “antisense strand” is used in reference to a nucleic acid strand that is complementary to the “sense” strand. In the normal operation of cellular metabolism, the sense strand of a DNA molecule is the strand that encodes polypeptides and/or other gene products. The sense strand serves as a template for synthesis of a messenger RNA (“mRNA”) transcript (an antisense strand) which, in turn, directs synthesis of any encoded gene product. Antisense nucleic acid molecules may be produced by any art-known methods, including synthesis by ligating the gene(s) of interest in a reverse orientation to a viral promoter which permits the synthesis of a complementary strand. Once introduced into a cell, this transcribed strand combines with natural sequences produced by the cell to form duplexes. These duplexes then block either the further transcription of the mRNA or its translation. The designations “negative” or (−) are also art-known to refer to the antisense strand, and “positive” or (+) are also art-known to refer to the sense strand.

For purposes of the present invention, “complementary” shall be understood to mean that a nucleic acid sequence forms hydrogen bond(s) with another nucleic acid sequence. A percent complementarity indicates the percentage of contiguous residues in a nucleic acid molecule which can form hydrogen bonds, i.e., Watson-Crick base pairing, with a second nucleic acid sequence, i.e., 5, 6, 7, 8, 9, 10 out of 10 being 50%, 60%, 70%, 80%, 90%, and 100% complementary. “Perfectly complementary” means that all the contiguous residues of a nucleic acid sequence form hydrogen bonds with the same number of contiguous residues in a second nucleic acid sequence.

The nucleic acids (such as one or more same or differen oligonucleotides or oligonucleotide derivatives) useful in the nanoparticle described herein can include from about 5 to about 1000 nucleic acids, and preferably relatively short polynucleotides, e.g., ranging in size preferably from about 8 to about 50 nucleotides in length (e.g., about 8, 9, 10, 11, 12, 13, 14, 15, 16, 17, 18, 19, 20, 21, 22, 23, 24, 25, 26, 27, 28, 29 or 30).

In one aspect of useful nucleic acids encapsulated within the nanoparticle described herein, oligonucleotides and oligodeoxynucleotides with natural phosphorodiester backbone or phosphorothioate backbone or any other modified backbone analogues include;

LNA (Locked Nucleic Acid);

PNA (nucleic acid with peptide backbone);

short interfering RNA (siRNA);

microRNA (miRNA);

nucleic acid with peptide backbone (PNA);

phosphorodiamidate morpholino oligonucleotides (PMO);

tricyclo-DNA;

decoy ODN (double stranded oligonucleotide);

catalytic RNA sequence (RNAi);

ribozymes;

aptamers;

spiegelmers (L-conformational oligonucleotides);

CpG oligomers, and the like, such as those disclosed at:

Tides 2002, Oligonucleotide and Peptide Technology Conferences, May 6-8, 2002, Las Vegas, Nev. and Oligonucleotide & Peptide Technologies, 18th & 19th Nov. 2003, Hamburg, Germany, the contents of which are incorporated herein by reference.

In another aspect of the nucleic acids encapsulated within the nanoparticle, oligonucleotides can optionally include any suitable art-known nucleotide analogs and derivatives, including those listed by Table 2, below:

TABLE 2
Representative Nucleotide Analogs And Derivatives

| | |
|---|---|
| 4-acetylcytidine | 5-methoxyaminomethyl-2-thiouridine |
| 5-(carboxyhydroxymethyl)uridine | beta, D-mannosylqueuosine |
| 2′-O-methylcytidine | 5-methoxycarbonylmethyl-2-thiouridine |
| 5-methoxycarbonylmethyluridine | 5-carboxymethylaminomethyl-2- |
| | thiouridine |
| 5-methoxyuridine | 5-carboxymethylaminomethyluridine |
| Dihydrouridine | 2-methylthio-N6-isopentenyladenosine |
| 2′-O-methylpseudouridine | N-[(9-beta-D-ribofuranosyl-2- |
| | methylthiopurine-6- |
| | yl)carbamoyl]threonine |
| D-galactosylqueuosine | N-[(9-beta-D-ribofuranosylpurine-6- |
| | y1)N-methylcarbamoyl]threonine |
| 2′-O-methylguanosine | uridine-5-oxyacetic acid-methylester |
| 2′-halo-adenosine | 2′-halo-cytidine |
| 2′-halo-guanosine | 2′-halo-thymine |
| 2′-halo-uridine | 2′-halo-methylcytidine |
| 2′-amino-adenosine | 2′-amino-cytidine |
| 2′-amino-guanosine | 2′-amino-thymine |
| 2′-amino-uridine | 2′-amino-methylcytidine |
| Inosine | uridine-5-oxyacetic acid |
| N6-isopentenyladenosine | Wybutoxosine |
| 1-methyladenosine | Pseudouridine |
| 1-methylpseudouridine | Queuosine |
| 1-methylguanosine | 2-thiocytidine |
| 1-methylinosine | 5-methyl-2-thiouridine |
| 2,2-dimethylguanosine | 2-thiouridine |
| 2-methyladenosine | 4-thiouridine |
| 2-methylguanosine | 5-methyluridine |
| 3-methylcytidine | N-[(9-beta-D-ribofuranosylpurine-6-y1)- |
| | carbamoyl]threonine |
| 5-methylcytidine | 2′-O-methyl-5-methyluridine |
| N6-methyladenosine | 2′-O-methyluridine |
| 7-methylguanosine | Wybutosine |
| 5-methylaminomethyluridine | 3-(3-amino-3-carboxy-propyl/uridine |
| Locked-adenosine | Locked-cytidine |
| Locked-guanosine | Locked-thymine |
| Locked-uridine | Locked-methylcytidine |

In one preferred aspect, the target oligonucleotides encapsulated in the nanoparticles include, for example, but are not limited to, oncogenes, pro-angiogenesis pathway genes, pro-cell proliferation pathway genes, viral infectious agent genes, and pro-inflammatory pathway genes.

In one preferred embodiment, the oligonucleotide encapsulated within the nanoparticle described herein is involved in targeting tumor cells or downregulating a gene or protein expression associated with tumor cells and/or the resistance of tumor cells to anticancer therapeutics. For example, antisense oligonucleotides for downregulating any art-known cellular proteins associated with cancer, e.g., BCL-2 can be used for the present invention. See U.S. patent application Ser. No. 10/822,205 filed Apr. 9, 2004, the contents of which are incorporated by reference herein. A non-limiting list of preferred therapeutic oligonucleotides includes antisense HIF1-α oligonucleotides, antisense survivin oligonucleotides, antisense ErbB3 oligonucleotides, antisense β-catenine oligonucleotides and antisense Bcl-2 oligonucleotides.

More preferably, the oligonucleotides according to the invention described herein include phosphorothioate backbone and LNA.

In one preferred embodiment, the oligonucleotide can be, for example, antisense survivin LNA, antisense ErbB3 LNA, or antisense HIF1-α LNA.

In another preferred embodiment, the oligonucleotide can be, for example, an oligonucleotide that has the same or substantially similar nucleotide sequence as does Genasense (a/k/a oblimersen sodium, produced by Genta Inc., Berkeley Heights, N.J.). Genasense is an 18-mer phosphorothioate antisense oligonucleotide, TCTCCCAGCGTGCGCCAT (SEQ ID NO: 4), that is complementary to the first six codons of the initiating sequence of the human bcl-2 mRNA (human bcl-2 mRNA is art-known, and is described, e.g., as SEQ ID NO: 19 in U.S. Pat. No. 6,414,134, incorporated by reference herein). The U.S. Food and Drug Administration (FDA) gave Genasense Orphan Drug status in August 2000.

Preferred embodiments contemplated include:

(i) antisense Survivin LNA, Oligo-1 (SEQ ID NO: 1)

5′-^{m}CT^{m}CAatccatgg^{m}CAGc-3′

where the upper case letter represents LNA, ^{m}C represents methylated cytosine, and the internucleoside linkage is phosphorothioate;

(ii) antisense ErbB3 LNA, Oligo-2 (SEQ ID NO: 2)

5′-TAGcctgtcactt^{m}CT^{m}C-3′

where the upper case letter represents LNA, ^{m}C represents methylated cytosine, and the internucleoside linkage is phosphorothioate;

(iii) Genasense, Oligo-4 (SEQ ID NO: 4)

5′-tctcccagcgtgcgcccat-3′

where the lower case letter represents DNA and internucleoside linkage is phosphorothioate;

(v) antisense HIF-1α LNA, Oligo-5 (SEQ ID NO: 5)

5′-TGGcaagcatccTGTa-3′

where the upper case letter represents LNA and internucleoside linkage is phosphorothioate; and

(vi) antisense Bcl2 siRNA:

SENSE

| | |
|---|---|
| 5′-gcaugcggccucuguuugadTdT-3′ | (SEQ ID NO: 6) |
| ANTISENSE | |
| 3′-dTdTcguacgccggagacaaacu-5′ | (SEQ ID NO: 7) |

where dT represents DNA.

LNA includes 2′-O,4′-C methylene bicyclonucleotide as shown below:

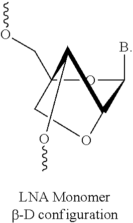

A scrambled antisense ErbB3 LNA, Oligo-3 (SEQ ID NO: 3) has the sequence of:

5′-TAGcttgtcccatt^{m}CT^{m}C-3′

where the upper case letter represents LNA, ^{m}C represents methylated cytosine, and the internucleoside linkage is phosphorothioate.

See detailed description of Survivin LNA disclosed in U.S. Patent Application Publication Nos. 2006/0154888, entitled “LNA Oligonucleotides and the Treatment of Cancer” and 2005/0014712, entitled “Oligomeric Compounds for the Modulation Survivin Expression”, the contents of each of which is incorporated herein by reference. See also U.S. Patent Application Publication Nos. 2004/0096848, entitled “Oligomeric Compounds for the Modulation HIF-1 Alpha Expression” and 2006/0252721, entitled “Potent LNA Oligonucleotides for Inhibition of HIF-1A Expression”, the contents of which are also incorporated herein by reference. See also, the contents of which are incorporated herein by reference in its entirety.

Examples of suitable target genes are described in PCT Publication No. WO 03/74654, PCT/US03/05028, and U.S. patent application Ser. No. 2007/0042983, the contents of which are incorporated by reference herein.

### F. Targeting Groups

Optionally/preferably, the nanoparticle compositions described herein further include a targeting ligand for a specific cell or tissue type. The targeting group can be attached to any component of a nanoparticle composition (preferably, fusogenic lipids and PEG-lipids) using a linker molecule, such as an amide, amido, carbonyl, ester, peptide, disulphide, silane, nucleoside, abasic nucleoside, polyether, polyamine, polyamide, peptide, carbohydrate, lipid, polyhydrocarbon, phosphate ester, phosphoramidate, thiophosphate, alkylphosphate, maleimidyl linker or photolabile linker. Any known techniques in the art can be used for conjugating a targeting group to any component of the nanoparticle composition without undue experimentation.

For example, targeting agents can be attached to the polymeric portion of PEG lipids to guide nanoparticles to the target area in vivo. The targeted delivery of the nanoparticle described herein enhances the cellular uptake of the nanoparticles encapsulating therapeutic nucleic acids to have better therapeutic efficacies. In certain aspects, some cell-penetrating peptides can be replaced with a variety of targeting peptides for targeted delivery to the tumor site.

In one preferred aspect of the invention, the targeting moiety, such as a single chain antibody (SCA) or single-chain antigen-binding antibody, monoclonal antibody, cell adhesion peptides such as RGD peptides and Selectin, cell penetrating peptides (CPPs) such as TAT, Penetratin and (Arg)₉, receptor ligands, targeting carbohydrate molecules or lectins allows nanoparticles to be specifically directed to targeted regions. See J Pharm Sci. 2006 September; 95(9):1856-72 Cell adhesion molecules for targeted drug delivery, the contents of which are incorporated herein by reference.

Preferred targeting moieties include single-chain antibodies (SCAs) or single-chain variable fragments of antibodies (sFv). The SCA contains domains of antibodies which can bind or recognize specific molecules of targeting tumor cells. In addition to maintaining an antigen binding site, a SCA conjugated to a PEG-lipid can reduce antigenicity and increase the half life of the SCA in the bloodstream.

The terms “single chain antibody” (SCA), “single-chain antigen-binding molecule or antibody” or “single-chain Fv” (sFv) are used interchangeably. The single chain antibody has binding affinity for the antigen. Single chain antibody (SCA) or single-chain Fvs can and have been constructed in several ways. A description of the theory and production of single-chain antigen-binding proteins is found in commonly assigned U.S. patent application Ser. No. 10/915,069 and U.S. Pat. No. 6,824,782, the contents of each of which are incorporated by reference herein.

Typically, SCA or Fv domains can be selected among monoclonal antibodies known by their abbreviations in the literature as 26-10, MOPC 315, 741F8, 520C9, McPC 603, D1.3, murine phOx, human phOx, RFL3.8 sTCR, 1A6, Se155-4,18-2-3,4-4-20,7A4-1, B6.2, CC49,3C2,2c, MA-15C5/K₁₂G_{O}, Ox, etc. (see, Huston, J. S. et al., Proc. Natl. Acad. Sci. USA 85:5879-5883 (1988); Huston, J. S. et al., SIM News 38(4) (Supp):11 (1988); McCartney, J. et al., ICSU Short Reports 10:114 (1990); McCartney, J. E. et al., unpublished results (1990); Nedelman, M. A. et al., J. Nuclear Med. 32 (Supp.):1005 (1991); Huston, J. S. et al., In: Molecular Design and Modeling: Concepts and Applications, Part B, edited by J. J. Langone, Methods in Enzymology 203:46-88 (1991); Huston, J. S. et al., In: Advances in the Applications of Monoclonal Antibodies in Clinical Oncology, Epenetos, A. A. (Ed.), London, Chapman & Hall (1993); Bird, R. E. et al., Science 242:423-426 (1988); Bedzyk, W. D. et al., J. Biol. Chem. 265:18615-18620 (1990); Colcher, D. et al., J. Nat. Cancer Inst. 82:1191-1197 (1990); Gibbs, R. A. et al., Proc. Natl. Acad. Sci. USA 88:4001-4004 (1991); Milenic, D. E. et al., Cancer Research 51:6363-6371 (1991); Pantoliano, M. W. et al., Biochemistry 30:10117-10125 (1991); Chaudhary, V. K. et al., Nature 339:394-397 (1989); Chaudhary, V. K. et al., Proc. Natl. Acad. Sci. USA 87:1066-1070 (1990); Batra, J. K. et al., Biochem. Biophys. Res. Comm. 171:1-6 (1990); Batra, J. K. et al., J. Biol. Chem. 265:15198-15202 (1990); Chaudhary, V. K. et al., Proc. Natl. Acad Sci. USA 87:9491-9494 (1990); Batra, J. K. et al., Mol. Cell. Biol. 11:2200-2205 (1991); Brinkmann, U. et al., Proc. Natl. Acad. Sci. USA 88:8616-8620 (1991); Seetharam, S. et al., J. Biol. Chem. 266:17376-17381 (1991); Brinkmann, U. et al., Proc. Natl. Acad. Sci. USA 89:3075-3079 (1992); Glockshuber, R. et al., Biochemistry 29:1362-1367 (1990); Skerra, A. et al., Bio/Technol. 9:273-278 (1991); Pack, P. et al., Biochemistry 31:1579-1534 (1992); Clackson, T. et al., Nature 352:624-628 (1991); Marks, J. D. et al., J. Mol. Biol. 222:581-597 (1991); Iverson, B. L. et al., Science 249:659-662 (1990); Roberts, V. A. et al., Proc. Natl. Acad. Sci. USA 87:6654-6658 (1990); Condra, J. H. et al., J. Biol. Chem. 265:2292-2295 (1990); Laroche, Y. et al., J. Biol. Chem. 266:16343-16349 (1991); Holvoet, P. et al., J. Biol. Chem. 266:19717-19724 (1991); Anand, N. N. et al., J. Biol. Chem. 266:21874-21879 (1991); Fuchs, P. et al., Biol Technol. 9:1369-1372 (1991); Breitling, F. et al., Gene 104:104-153 (1991); Seehaus, T. et al., Gene 114:235-237 (1992); Takkinen, K. et al., Protein Engng. 4:837-841 (1991); Dreher, M. L. et al., J. Immunol. Methods 139:197-205 (1991); Mottez, E. et al., Eur. J. Immunol. 21:467-471 (1991); Traunecker, A. et al., Proc. Natl. Acad. Sci. USA 88:8646-8650 (1991); Traunecker, A. et al., EMBO J. 10:3655-3659 (1991); Hoo, W. F. S. et al., Proc. Natl. Acad. Sci. USA 89:4759-4763 (1993)). Each of the foregoing publications is incorporated herein by reference.

A non-limiting list of targeting groups includes vascular endothelial cell growth factor, FGF2, somatostatin and somatostatin analogs, transferrin, melanotropin, ApoE and ApoE peptides, von Willebrand's Factor and von Willebrand's Factor peptides, adenoviral fiber protein and adenoviral fiber protein peptides, PD1 and PD1 peptides, EGF and EGF peptides, RGD peptides, folate, etc. Other optional targeting agents appreciated by artisans in the art can be also employed in the nanoparticles described herein.

In one preferred embodiment, the targeting agents useful for the nanoparticle described herein include single chain antibody (SCA), RGD peptides, selectin, TAT, penetratin, (Arg)₉, folic acid, etc., and some of the preferred structures of these agents are:

| C-TAT: | |
|---|---|
| CYGRKKRRQRRR; | (SEQ ID NO: 8) |
| C-(Arg)₉: | |
| CRRRRRRRRR; | (SEQ ID NO: 9) |

RGD can be linear or cyclic:

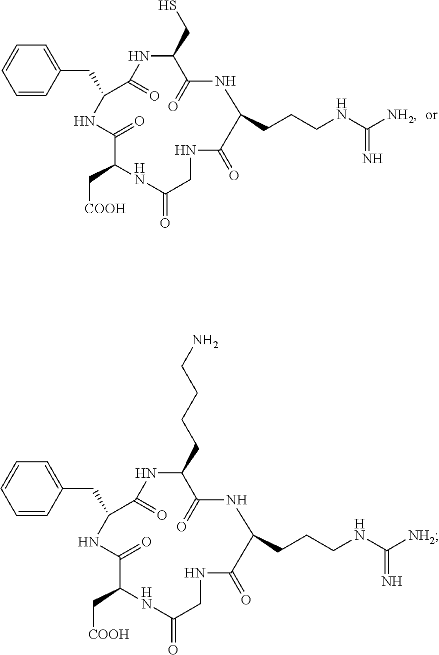

and

Folic acid is a residue of

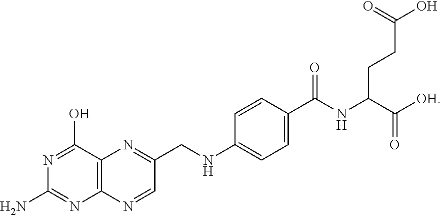

Arg₉ can include a cysteine for conjugating such as CRRRRRRRRR and TAT can add an additional cysteine at the end of the peptide such as CYGRKKRRQRRRC (SEQ ID NO: 10).

For purpose of the current invention, the abbreviations used in the specification and figures represent the following structures:

(i) C-diTAT
(SEQ ID NO: 11) = CYGRKKRRQRRRYGRKKRRQRRR-NH₂;
(ii) Linear RGD
(SEQ ID NO: 12) = RGDC;
(iii) Cyclic RGD
(SEQ ID NO: 13) = c-RGDFC;
(iv) RGD-TAT
(SEQ ID NO: 14) = CYGRKKRRQRRRGGGRGDS-NH₂;
and
(v) Arg₉.
(SEQ ID NO: 15)

Alternatively, the targeting group includes sugars and carbohydrates such as galactose, galactosamine, and N-acetyl galactosamine; hormones such as estrogen, testosterone, progesterone, glucocortisone, adrenaline, insulin, glucagon, cortisol, vitamin D, thyroid hormone, retinoic acid, and growth hormones; growth factors such as VEGF, EGF, NGF, and PDGF; neurotransmitters such as GABA, glutamate, acetylcholine; NOGO; inostitol triphosphate; epinephrine; norepinephrine; nitric oxide, peptides, vitamins such as folate and pyridoxine, drugs, antibodies and any other molecule that can interact with a receptor in vivo or in vitro.

### G. Preparation of Cationic Lipids of Formula (I)

Generally, the methods of preparing cationic lipids of Formula (I) described herein include reacting an amine-containing cholesterol (functionalized cholesterol) with 1H-pyrazole-1-carboxamidine to provide a guanidinium moiety. The amine linked to cholesterol can be a primary and/or secondary amine and the amines in 1H-pyrazole-1-carboxamidine can be unsubstituted or substituted.

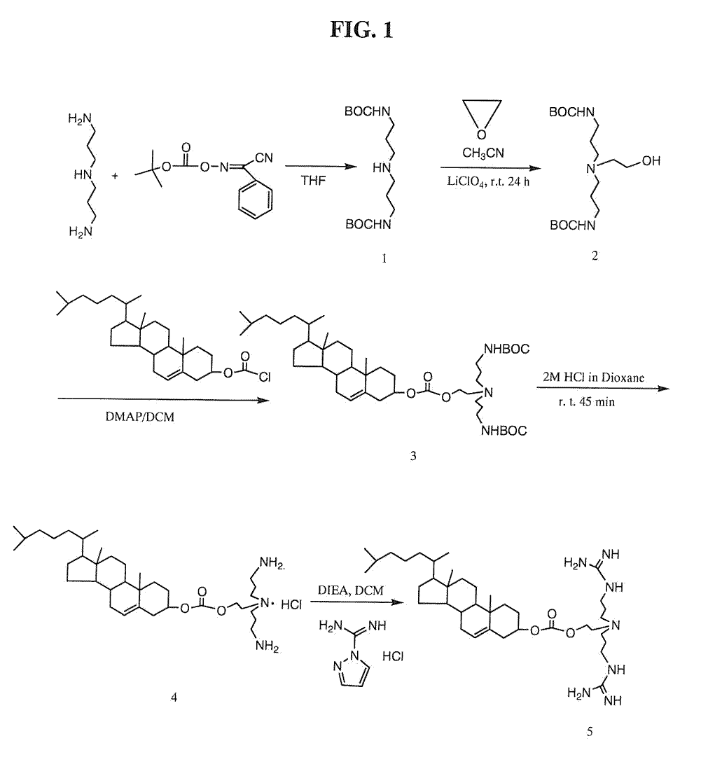

One example of the preparation of the cholesteryl cationic lipid described herein is shown in FIG. 1. Terminal primary amines of N-(3-aminopropyl)-1,3-propanediamine were selectively protected with Boc groups, followed by reacting the secondary amine of bis-N-Boc-(3-aminopropyl)-1,3-propanediamine (compound 2) with an epoxide to prepare compound 2 containing a nucleophile, OH. An activated cholesterol carbonate such as cholesteryl chloroformate, cholesteryl NHS carbonate, or cholesteryl PNP carbonate, can react with the nucleophile OH to provide compound 3. By deprotection of the Boc moieties in an acidic condition, an amine containing cholesterol (compound 4) was prepared. The amines of compound 4 reacted with 1H-pyrazole-1-carboxamidine to provide a cholesteryl cationic lipid containing guanidinium moieties (compound 5).

In another embodiment, attachment of an amine-containing compound to a cholesterol can be carried out using standard organic synthetic techniques in the presence of a base, using coupling agents known to those of ordinary skill in the art such as 1,3-diisopropylcarbodiimide (DIPC), dialkyl carbodiimides, 2-halo-1-alkylpyridinium halides, 1-(3-dimethylaminopropyl)-3-ethyl carbodiimide (EDC), propane phosphonic acid cyclic anhydride (PPACA) and phenyl dichlorophosphates.

Alternatively, when a cholesterol or amine-containing compound is activated with a leaving group such as NHS, PNP, or chloroformate, the reaction can be carried out in the presence of a base without a coupling agent.

Generally, the cationic lipids of Formula (I) described herein are preferably prepared by reacting an activated cholesterol with an amine containing nucleophile such as compound 2 in the presence of a base such as DMAP or DIEA. Preferably, the reaction is carried out in an inert solvent such as methylene chloride, chloroform, toluene, DMF or mixtures thereof. The reaction is also preferably conducted in the presence of a base, such as DMAP, DIEA, pyridine, triethylamine, etc. at a temperature from about −4° C. to about 70° C. (e.g. −4° C. to about 50° C.). In one preferred embodiment, the reaction is performed at a temperature from about 0° C. to about 25° C. or 0° C. to about room temperature.

Removal of a protecting group from an amine-containing compound, such as compound 3, can be carried out with a strong acid such as trifluoroacetic acid (TFA), HCl, sulfuric acid, etc., or by catalytic hydrogenation, radical reaction, etc. In one embodiment, the deprotection of a Boc group is carried out with HCl solution in dioxane. The deprotection reaction can be carried out at a temperature from about −4° C. to about 50° C. Preferably, the reaction is carried out at a temperature from about 0° C. to about 25° C. or to room temperature. In another embodiment, the deprotection of a Boc group is carried out at room temperature.

Conversion of an amine to a guanidine group is carried out by reacting an amine linked to a cholesterol (e.g., the amines of compound 4) with 1H-pyrazole-1-carboxamidine in an inert solvent such as methylene chloride, chloroform, DMF or mixtures thereof. Other reagents, such as N-BOC-1H-pyrazole-1-carboxamidine or N,N′-Di-(tert-butoxycarbonyl)thiourea and a coupling reagent can be also used to convert an amine to a guanidine moiety. The coupling agents known to those of ordinary skill in the art, such as 1,3-diisopropylcarbodiimide (DIPC), diallyl carbodiimides, 2-halo-1-alkylpyridinium halides, 1-(3-dimethylaminopropyl)-3-ethyl carbodiimide (EDC), propane phosphonic acid cyclic anhydride (PPACA) and phenyl dichlorophosphates, can be employed in the reaction. The reaction is preferably conducted in the presence of a base, such as DMAP, DIEA, pyridine, triethylamine, etc. at a temperature from about −4° C. to about 50° C. In one preferred embodiment, the reaction is performed at a temperature from about 0° C. to about 25° C. or to room temperature.

### H. Nanoparticle Compositions/Formulations

The nanoparticle composition described herein contains a cationic lipid of Formula (I), a fusogenic lipid and a PEG-lipid.

In one preferred aspect, the nanoparticle composition includes cholesterol.

In a further aspect of the present invention, the nanoparticle composition described herein may contain additional art-known cationic lipids. The nanoparticle composition containing a mixture of different fusogenic lipids (non-cationic lipids) and/or a mixture of different PEG-lipids are also contemplated.

In another aspect, the nanoparticle composition described herein contains the cationic lipid of Formula (I) described herein in a molar ratio ranging from about 10% to about 99.9% of the total lipid (pharmaceutical carrier) present in the nanoparticle composition.

The cationic lipid component can range from about 2% to about 60%, from about 5% to about 50%, from about 10% to about 45%, from about 15% to about 25%, or from about 30% to about 40% of the total lipid present in the nanoparticle composition.

In one particular embodiment, the cationic lipid is present in amounts of from about 15 to about 25% (i.e., 15, 16, 17, 18, 19, 20, 21, 22, 23, 24 or 25%) of the total lipid present in the nanoparticle composition.

In another aspect of the nanoparticle composition described herein, the compositions contain a fusogenic/non-cationic lipid, including cholesterol and/or noncholesterol-based fusogenic lipid, in a molar ratio of from about 20% to about 85%, from about 25% to about 85%, from about 60% to about 80% (e.g., 65, 75, 78, or 80%) of the total lipid present in the nanoparticle composition. In one particular embodiment, a total fusogenic/non-cationic lipid is about 80% of the total lipid present in the nanoparticle composition.

In yet another aspect, a noncholesterol-based fusogenic/non-cationic lipid is present in a molar ratio of from about 25 to about 78% (25, 35, 47, 60, or 78%), or from about 60 to about 78% of the total lipid present in the nanoparticle composition. In one particular embodiment, a noncholesterol-based fusogenic/non-cationic lipid is about 60% of the total lipid present in the nanoparticle composition.

In yet another aspect, the nanoparticle composition includes cholesterol, in addition to non-cholesterol fusogenic lipid, in a molar ratio ranging from about 0% to about 60%, from about 10% to about 60%, or from about 20% to about 50% (e.g., 20, 30, 40 or 50%) of the total lipid present in the nanoparticle composition. In one particular embodiment, cholesterol is about 20% of the total lipid present in the nanoparticle composition.

In yet another aspect of the invention, the PEG-lipid contained in the nanoparticle composition ranges in a molar ratio of from about 0.5% to about 20%, from about 1.5% to about 18% of the total lipid present in the nanoparticle composition. In one embodiment of the nanoparticle composition, the PEG lipid is included in a molar ratio of from about 2% to about 10% (e.g., 2, 3, 4, 5, 6, 7, 8, 9, or 10%) of the total lipid. For example, a total PEG lipid is about 2% of the total lipid present in the nanoparticle composition.

### I. Preparation of Nanoparticles

The nanoparticle described herein can be prepared by any art-known process without undue experimentation. For example, the nanoparticle can be prepared by providing nucleic acids such as oligonucleotides in an aqueous solution (or an aqueous solution without nucleic acids for comparison study) in a first reservoir, providing an organic lipid solution containing the nanoparticle composition described herein in a second reservoir, and mixing the aqueous solution with the organic lipid solution such that the organic lipid solution mixes with the aqueous solution to produce nanoparticles encapsulating the nucleic acids. Details of the process are described in U.S. Patent Publication No. 2004/0142025, the contents of which are incorporated herein by reference.

Alternatively, the nanoparticles described herein can be prepared by using any methods known in the art including, e.g., a detergent dialysis method or a modified reverse-phase method which utilizes organic solvents to provide a single phase during mixing the components. In a detergent dialysis method, nucleic acids (i.e., LNA, siRNA, etc.) are contacted with a detergent solution of cationic lipids to form a coated nucleic acid complex.

In one embodiment of the invention, the cationic lipids and nucleic acids such as oligonucleotides are combined to produce a charge ratio of from about 1:1 to about 20:1, from about 1:1 to about 12:1, and more preferably in a ratio of from about 2:1 to about 6:1. Alternatively, the nitrogen to phosphate (N/P) ratio of the nanoparticle composition ranges from about 2:1 to about 5:1, (i.e., 2.5:1).

In another embodiment, the nanoparticle described herein can be prepared by using a dual pump system. Generally, the process includes providing an aqueous solution containing nucleic acids in a first reservoir and a lipid solution containing the nanoparticle composition described in a second reservoir. The two solutions are mixed by using a dual pump system to provide nanoparticles. The resulting mixed solution is subsequently diluted with an aqueous buffer and the nanoparticles formed can be purified and/or isolated by dialysis. The nanoparticles can be further processed to be sterilized by filtering through a 0.22 μm filter.

The nanoparticles containing nucleic acids range from about 5 to about 300 nm in diameter. Preferably, the nanoparticles have a median diameter of less than about 150 nm (e.g., about 50-150 nm), more preferably a diameter of less than about 100 nm, by the measurement using the Dynamic Light Scattering technique (DLS). A majority of the nanoparticles have a median diameter of about 30 to 100 nm (e.g., 59.5, 66, 68, 76, 80, 93, 96 nm), preferably about 60 to about 95 nm. Artisans will appreciate that the measurement using other art-known techniques such as TEM may provide a median diameter number decreased by half, as compared to the DLS technique. The nanoparticles of the present invention are substantially uniform in size as shown by polydispersity.

Optionally, the nanoparticles can be sized by any methods known in the art. The size can be controlled as desired by artisans. The sizing may be conducted in order to achieve a desired size range and relatively narrow distribution of nanoparticle sizes. Several techniques are available for sizing the nanoparticles to a desired size. See, for example, U.S. Pat. No. 4,737,323, the contents of which are incorporated herein by reference.

The present invention provides methods for preparing serum-stable nanoparticles such that nucleic acids (e.g., LNA or siRNA) are encapsulated in a lipid multi-lamellar structure (i.e. a lipid bilayer) and are protected from degradation. The nanoparticles described herein are stable in an aqueous solution. Nucleic acids included in the nanoparticles are protected from nucleases present in the body fluid.

Additionally, the nanoparticles prepared according to the present invention are preferably neutral or positively-charged at physiological pH.

The nanoparticle or nanoparticle complex prepared using the nanoparticle composition described herein includes: (i) a cationic lipid of Formula (I); (ii) a neutral lipid/fusogenic lipid; (iii) a PEG-lipid and (iv) nucleic acids such as an oligonucleotide.

In one embodiment, the nanoparticle composition includes a mixture of

a cationic lipid of Formula (I), a diacylphosphatidylethanolamine, a PEG conjugated to phosphatidylethanolamine (PEG-PE), and cholesterol;

a cationic lipid of Formula (I), a diacylphosphatidylcholine, a PEG conjugated to phosphatidylethanolamine (PEG-PE), and cholesterol;

a cationic lipid of Formula (I), a diacylphosphatidylethanolamine, a diacylphosphatidylcholine, a PEG conjugated to phosphatidylethanolamine (PEG-PE), and cholesterol;

a cationic lipid of Formula (I), a diacylphosphatidylethanolamine, a PEG conjugated to ceramide (PEG-Cer), and cholesterol; or

a cationic lipid of Formula (I), a diacylphosphatidylethanolamine, a PEG conjugated to phosphatidylethanolamine (PEG-PE), a PEG conjugated to ceramide (PEG-Cer), and cholesterol.

Additional nanoparticle compositions can be prepared by modifying compositions containing art-known cationic lipid(s). Nanoparticle compositions containing art-known cationic lipid(s) can be modified by replacing art-known cationic lipids with a cationic lipid of Formula (I) and/or adding a cationic lipid of Formula (I). See art-known compositions described in Table IV of US Patent Application Publication No. 2008/0020058, the contents of which are incorporated herein by reference.

A non-limiting list of nanoparticle compositions for the preparation of nanoparticles is set forth in Table 3.

TABLE 3

| Sample | | | |
|---|---|---|---|
| No. | Nanoparticle Composition | Molar Ratio | Oligo |
| 1 | Compd 5: DOPE:DSPC:Chol:DSPE-PEG | 15:15:20:40:10 | Oligo-1 |
| 2 | Compd 5: DOPE:DSPC:Chol:DSPE-PEG | 15:5:20:50:10 | Oligo-1 |
| 3 | Compd 5: DOPE:DSPC:Chol:DSPE-PEG | 25:15:20:30:10 | Oligo-1 |
| 4 | Compd 5: EPC:Chol:DSPE-PEG | 20:47:30: 3 | Oligo-1 |
| 5 | Compd 5: DOPE:Chol:DSPE-PEG | 17:60:20:3 | Oligo-1 |
| 6 | Compd 5: DOPE:DSPE-PEG | 20:78:2 | Oligo-1 |
| 7 | Compd 5: DOPE:Chol:C16mPEG-Ceramide | 17:60:20:3 | Oligo-2 |
| 8 | Compd 5: DOPE:Chol:DSPE-PEG:C16mPEG-Ceramide | 18:60:20:1:1 | Oligo-2 |

In one embodiment, the molar ratio of a cationic lipid (compound 5):DOPE:cholesterol:PEG-DSPE:C16mPEG-Ceramide in the nanoparticle is in a molar ratio of about 18%:60%:20%:1%:1%, respectively based the total lipid present in the nanoparticle composition (Sample No. 8).

In another embodiment, the nanoparticle contains a cationic lipid (compound 5), DOPE, cholesterol and C16mPEG-Ceramide in a molar ratio of about 17%:60%:20%:3% of the total lipid present in the nanoparticle composition (Sample No. 7).

These nanoparticle compositions preferably contain a cationic lipid having the structure:

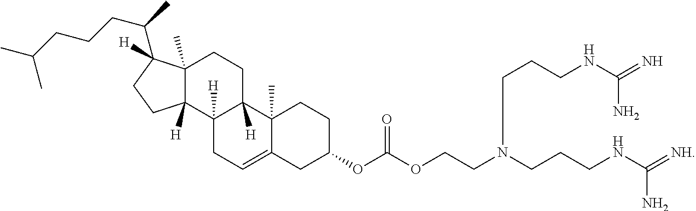

The molar ratio as used herein refers to the amount relative to the total lipid present in the nanoparticle composition.

### J. Methods of Treatment

The nanoparticles described herein can be employed in the treatment for preventing, inhibiting, reducing or treating any trait, disease or condition that is related to or responds to the levels of target gene expression in a cell or tissue, alone or in combination with other therapies. The method includes administering the nanoparticle described herein to a mammal in need thereof.

One aspect of the present invention provides methods of introducing or delivering therapeutic nucleic acids such as oligonucleotides into a mammalian cell in vivo and/or in vitro. The method according to the present invention includes contacting a cell with the nanoparticle described herein. The delivery can be made in vivo as part of a suitable pharmaceutical composition or directly to the cells in an ex vivo environment.

In another aspect, the present invention is useful for introducing oligonucleotides to a mammal. The nanoparticles described herein can be administered to a mammal, preferably human.

In yet another aspect, the present invention preferably provides methods of inhibiting or downregulating (or modulating) a gene expression in mammalian cells or tissues. The downregulation or inhibition of gene expression can be achieved in vivo, ex vivo and/or in vitro. The methods include contacting human cells or tissues with nanoparticles encapsulating nucleic acids described herein or administering the nanoparticles in a mammal in need thereof. Once the contacting has occurred, successful inhibition or down-regulation of gene expression such as in mRNA or protein levels shall be deemed to occur when at least about 10%, preferably at least about 20% or higher (e.g., at least about 25%, 30%, 40%, 50%, 60%) is realized in vivo, ex vivo or in vitro when compared to that observed in the absence of the nanoparticles described herein.

For purposes of the present invention, “inhibiting” or “down-regulating” shall be understood to mean that the expression of a target gene, or level of RNAs or equivalent RNAs encoding one or more protein subunits, or activity of one or more protein subunits, such as ErbB3, HIF-1α, Survivin and BCL2, is reduced when compared to that observed in the absence of the nanoparticles described herein.

In one preferred embodiment, a target gene includes, for example, but is not limited to, oncogenes, pro-angiogenesis pathway genes, pro-cell proliferation pathway genes, viral infectious agent genes, and pro-inflammatory pathway genes.

Preferably, gene expression of a target gene is inhibited in cancer cells or tissues, for example, brain, breast, colorectal, gastric, lung, mouth, pancreatic, prostate, skin or cervical cancer cells. The cancer cells or tissues can be from one or more of the following: solid tumors, lymphomas, small cell lung cancer, acute lymphocytic leukemia (ALL), pancreatic cancer, glioblastoma, ovarian cancer, gastric cancer, breast cancer, colorectal cancer, prostate cancer, cervical cancer, ovarian cancer, brain tumors, KB cancer, lung cancer, colon cancer, epidermal cancer, etc.

In one particular embodiment, the nanoparticles according to the method described herein includes, for example, antisense bcl-2 oligonucleotides, antisense HIF-1α oligonucleotides, antisense Survivin oligonucleotides and antisense ErbB3 oligonucleotides.

The therapy contemplated herein uses nucleic acids encapsulated in the aforementioned nanoparticle. In one embodiment, therapeutic nucleotides containing eight or more consecutive antisense nucleotides can be employed in the treatment.

In one particular treatment, the nanoparticles including oligonucleotides (SEQ ID NO. 1, SEQ ID NO: 2, SEQ ID NO: 4, and SEQ ID NO: 5) can be used.

Alternatively, there are also provided methods of treating a mammal. The methods include administering an effective amount of a pharmaceutical composition containing a nanoparticle described herein to a patient in need thereof. The efficacy of the methods would depend upon efficacy of the nucleic acids for the condition being treated. The present invention provides methods of treatment for various medical conditions in mammals. The methods include administering, to the mammal in need of such treatment, an effective amount of a nanoparticle containing encapsulated therapeutic nucleic acids. The nanoparticles described herein are useful for, among other things, treating diseases for example, but not limited to, cancer, inflammatory disease, and autoimmune disease.

In one embodiment, there are also provided methods of treating a patient having a malignancy or cancer, comprising administering an effective amount of a pharmaceutical composition containing the nanoparticle described herein to a patient in need thereof. The cancer being treated can be one or more of the following: solid tumors, lymphomas, small cell lung cancer, acute lymphocytic leukemia (ALL), pancreatic cancer, glioblastoma, ovarian cancer, gastric cancers, colorectal cancer, prostate cancer, cervical cancer, brain tumors, KB cancer, lung cancer, colon cancer, epidermal cancer, etc.

The nanoparticles are useful for treating neoplastic disease, reducing tumor burden, preventing metastasis of neoplasms and preventing recurrences of tumor/neoplastic growths in mammals by downregulating gene expression of a target gene. For example, the nanoparticles are useful in the treatment of metastatic disease (i.e. cancer with metastasis into the liver).

In yet another aspect, the present invention provides methods of inhibiting the growth or proliferation of cancer cells in vivo or in vitro. The methods include contacting cancer cells with the nanoparticle described herein. In one embodiment, the present invention provides methods of inhibiting the growth of cancer in vivo or in vitro wherein the cells express ErbB3 gene. Cancer cells contact the antisense ErbB3 oligonucleotides released from the nanoparticles described herein. The antisense strand complementary to mRNA expressed from human ErbB3 gene inhibits growth of the cancer cells and reduces expression of the ErbB3 gene in cancer cells such as lymphoma or leukemia cells. Alternatively, the present invention provides methods of modulating apoptosis in cancer cells. The method includes contacting cells with the nanoparticle described herein.

In yet another aspect, there are also provided methods of increasing the sensitivity of cancer cells or tissues to chemotherapeutic agents in vivo or in vitro. In one particular aspect, the methods include introducing an oligonucleotide (e.g. antisense oligonucleotides including LNA) encapsulated in the nanoparticle described herein to cancer cells to reduce gene (e.g., survivin, HIF-1α or ErbB3) expression in the cancer cells or tissues, wherein the antisense oligonucleotide binds to mRNA and reduces gene expression.

In yet another aspect, there are provided methods of killing tumor cells in vivo or in vitro. The methods include introducing the nanoparticles described herein to tumor cells to reduce gene expression such as ErbB3 gene and contacting the tumor cells with an amount of at least one chemotherapeutic agent sufficient to kill a portion of the tumor cells. Thus, the portion of tumor cells killed can be greater than the portion which would have been killed by the same amount of the chemotherapeutic agent in the absence of the nanoparticles described herein.

In a further aspect of the invention, a chemotherapeutic agent can be used in combination, simultaneously or sequentially, in the methods employing the nanoparticles described herein. The nanoparticles described herein can be administered prior to or concurrently with the chemotherapeutic agent or after the administration of the chemotherapeutic agent.

Alternatively, the nanoparticle composition described herein can be used to deliver a pharmaceutically active compound, preferably having a negative charge or a neutral charge to a mammal. The nanoparticle encapsulating pharmaceutically active compounds can be administered to a mammal in need thereof. The pharmaceutically active compounds include small molecular weight molecules. Typically, the pharmaceutically active compounds have a molecular weight of less than about 1,500 daltons (i.e., less than 1,000 daltons).

In a further embodiment, the compounds described herein can be used to deliver nucleic acids, a pharmaceutically active agent, or in a combination thereof.

In yet a further embodiment, the nanoparticle associated with the treatment can contain a mixture of one or more therapeutic nucleic acids (either the same or different, for example, the same or different oligonucleotides containing LNA) and pharmaceutically active agents for synergistic application.

### K. Pharmaceutical Compositions/Formulations of Nanoparticles

Pharmaceutical compositions/formulations including the nanoparticles described herein may be formulated in conjunction with one or more physiologically acceptable carriers comprising excipients and auxiliaries which facilitate processing of the active compounds into preparations which can be used pharmaceutically. Proper formulation is dependent upon the route of administration chosen, i.e. whether local or systemic treatment is treated.

Suitable forms, in part, depend upon the use or the route of entry, for example oral, transdermal, or injection. Factors for considerations known in the art for preparing proper formulations include, but are not limited to, toxicity and any disadvantages that would prevent the composition or formulation from exerting its effect.

Administration of pharmaceutical compositions of nanoparticles described herein may be oral, pulmonary, topical (e.g., epidermal, transdermal, ophthalmic and mucous membranes including vaginal and rectal delivery), or parenteral including intravenous, intraarterial, subcutaneous, intraperitoneal or intramuscular injection or infusion.

In one preferred embodiment, the nanoparticles containing therapeutic oligonucleotides are administered intravenously (i.v.), intraperitoneally (i.p.) or as a bolus injection. Parenteral routes are preferred in many aspects of the invention.

For injection, including, without limitation, intravenous, intramuscular and subcutaneous injection, the nanoparticles of the invention may be formulated in aqueous solutions, preferably in physiologically compatible buffers such as physiological saline buffer or polar solvents including, without limitation, a pyrrolidone or dimethylsulfoxide.

The nanoparticles may also be formulated for bolus injection or for continuous infusion. Formulations for injection may be presented in unit dosage form, e.g., in ampoules or in multi-dose containers. Useful compositions include, without limitation, suspensions, solutions or emulsions in oily or aqueous vehicles, and may contain adjuncts such as suspending, stabilizing and/or dispersing agents. Pharmaceutical compositions for parenteral administration include aqueous solutions of a water soluble form. Aqueous injection suspensions may contain substances that modulate the viscosity of the suspension, such as sodium carboxymethyl cellulose, sorbitol, or dextran. Optionally, the suspension may also contain suitable stabilizers and/or agents that increase the concentration of the nanoparticles in the solution. Alternatively, the nanoparticles may be in powder form for constitution with a suitable vehicle, e.g., sterile, pyrogen-free water, before use.

For oral administration, the nanoparticles described herein can be formulated by combining the nanoparticles with pharmaceutically acceptable carriers well-known in the art. Such carriers enable the nanoparticles of the invention to be formulated as tablets, pills, lozenges, dragees, capsules, liquids, gels, syrups, pastes, slurries, solutions, suspensions, concentrated solutions and suspensions for diluting in the drinking water of a patient, premixes for dilution in the feed of a patient, and the like, for oral ingestion by a patient. Pharmaceutical preparations for oral use can be made using a solid excipient, optionally grinding the resulting mixture, and processing the mixture of granules, after adding other suitable auxiliaries if desired, to obtain tablets or dragee cores. Useful excipients are, in particular, fillers such as sugars, including lactose, sucrose, mannitol, or sorbitol, cellulose preparations such as, for example, maize starch, wheat starch, rice starch and potato starch and other materials such as gelatin, gum tragacanth, methyl cellulose, hydroxypropylmethylcellulose, sodium carboxymethylcellulose, and/or polyvinylpyrrolidone (PVP). If desired, disintegrating agents may be added, such as cross-linked polyvinyl pyrrolidone, agar, or alginic acid. A salt such as sodium alginate may also be used.

For administration by inhalation, the nanoparticles of the present invention can conveniently be delivered in the form of an aerosol spray using a pressurized pack or a nebulizer and a suitable propellant.

The nanoparticles may also be formulated in rectal compositions such as suppositories or retention enemas, using, e.g., conventional suppository bases such as cocoa butter or other glycerides.

In addition to the formulations described previously, the nanoparticles may also be formulated as depot preparations. Such long acting formulations may be administered by implantation (for example, subcutaneously or intramuscularly) or by intramuscular injection. A nanoparticle of this invention may be formulated for this route of administration with suitable polymeric or hydrophobic materials (for instance, in an emulsion with a pharmacologically acceptable oil), with ion exchange resins, or as a sparingly soluble derivative such as, without limitation, a sparingly soluble salt.

Additionally, the nanoparticles may be delivered using a sustained-release system, such as semi-permeable matrices of solid hydrophobic polymers containing the nanoparticles. Various sustained-release materials have been established and are well known by those skilled in the art.

In addition, antioxidants and suspending agents can be used in the pharmaceutical compositions of the nanoparticles described herein.

### L. Dosages

Determination of a therapeutically effective amount is well within the capability of those skilled in the art, especially in light of the disclosure herein.

For any therapeutic nucleic acids used in the methods of the invention, the therapeutically effective amount can be estimated initially from in vitro assays. Then, the dosage can be formulated for use in animal models so as to achieve a circulating concentration range that includes the effective dosage. Such information can then be used to more accurately determine dosages useful in patients.

The amount of the pharmaceutical composition that is administered will depend upon the potency of the nucleic acids included therein. Generally, the amount of the nanoparticles containing nucleic acids used in the treatment is that amount which effectively achieves the desired therapeutic result in mammals. Naturally, the dosages of the various nanoparticles will vary somewhat depending upon the nucleic acids (or pharmaceutically active agents) encapsulated therein (oligonucleotides such as antisense LNA molecules). In addition, the dosage, of course, can vary depending upon the dosage form and route of administration. In general, however, the nucleic acids encapsulated in the nanoparticles described herein can be administered in amounts ranging from about 0.1 mg/kg/dose to about 1 g/kg/dose, preferably from about 1 to about 500 mg/kg/dose and more preferably from 1 to about 100 mg/kg/dose (i.e., from about 2 to about 60 mg/kg/dose). The antisense oligonucleotide administered in the therapy can range in an amount of from about 4 to about 25 mg/kg/dose. For example, the treatment protocol includes administering an antisense oligonucleotide ranging from about 0.1 mg/kg/week to about 1 g/kg/week, preferably from about 1 to about 500 mg/kg/week and more preferably from 1 to about 100 mg/kg/week (i.e., from about 2 to about 60 mg/kg/week).

In one embodiment, the protocol includes administering an antisense oligonucleotide in an amount of about 4 to about 18 mg/kg/dose weekly, or about 4 to about 9.5 mg/kg/dose weekly.

In one particular embodiment, the treatment protocol includes an antisense oligonucleotide in an amount of about 4 to about 18 mg/kg/dose weekly for 3 weeks in a six week cycle (i.e. about 8 mg/kg/dose). Another particular embodiment includes about 4 to about 9.5 mg/kg/dose weekly (i.e., about 8 or 4.1 mg/kg/dose).

The range set forth above is illustrative and those skilled in the art will determine the optimal dosing based on clinical experience and the treatment indication. Moreover, the exact formulation, route of administration and dosage can be selected by the individual physician in view of the patient's condition. Additionally, toxicity and therapeutic efficacy of the compounds described herein can be determined by standard pharmaceutical procedures in cell cultures or experimental animals using methods well-known in the art.

Alternatively, an amount of from about 0.1 mg to about 140 mg/kg/day (0.1 to 100 mg/kg/day) can be used in the treatment depending on potency of the nucleic acids. Dosage unit forms generally range from about 1 mg to about 500 mg of an active agent, oligonucleotides.

In one embodiment, the treatment of the present invention includes administering the oligonucleotide encapsulated within the nanoparticles described herein in an amount of from about 0.1 to about 50 mg/kg/dose, such as from about 0.5 to about 45 mg/kg/dose (e.g. either in a single or multiple dose regime) to a mammal.

Alternatively, the delivery of the oligonucleotide encapsulated within the nanoparticles described herein includes contacting a concentration of oligonucleotides of from about 0.1 to about 1000 nM, preferably from about 10 to about 1500 nM (i.e. from about 30 to about 1000 nM) with tumor cells or tissues in vivo, ex vivo or in vitro.

The compositions may be administered once daily or divided into multiple doses which can be given as part of a multi-week treatment protocol. The precise dose will depend on the stage and severity of the condition, the susceptibility of the disease such as tumor to the nucleic acids, and the individual characteristics of the patient being treated, as will be appreciated by one of ordinary skill in the art.

In all aspects of the invention where nanoparticles are administered, the dosage amount mentioned is based on the amount of oligonucleotide molecules rather than the amount of nanoparticles administered.

It is contemplated that the treatment will be given for one or more days until the desired clinical result is obtained. The exact amount, frequency and period of administration of the nanoparticles encapsulating therapeutic nucleic acids (or pharmaceutically active agents) will vary, of course, depending upon the sex, age and medical condition of the patent as well as the severity of the disease as determined by the attending clinician.

Still further aspects include combining the nanoparticles of the present invention described herein with other anticancer therapies for synergistic or additive benefit.

### EXAMPLES

The following examples serve to provide further appreciation of the invention but are not meant in any way to restrict the effective scope of the invention.

In the examples, all synthesis reactions are run under an atmosphere of dry nitrogen or argon. N-(3-aminopropyl)-1,3-propanediamine, BOC-ON, ethylene oxide, LiOCl₄, cholesterol and 1H-pyrazole-1-carboxamidine.HCl were purchased from Aldrich. All other reagents and solvents were used without further purification. An LNA-containing oligonucleotides such as Oligo-1 targeting survivin gene, Oligo-2 targeting ErbB3 gene and Oligo-3 (scrambled Oligo-2) were prepared in house and their sequences are described in Table 4. The internucleoside linkage in the oligonucleotides includes phosphorothioate, ^{m}C represents methylated cytosine, and the upper case letters indicate LNA.

TABLE 4

| LNA Oligo | Sequence |
|---|---|
| Oligo-1 (SEQ ID NO: 1) | 5′-^{m}CT^{m}CAatccatgg^{m}CAGc-3′ |
| Oligo-2 (SEQ ID NO: 2) | 5′-TAGcctgtcactt^{m}CT^{m}C-3′ |
| Oligo-3 (SEQ ID NO: 3) | 5′-TAGcttgtcccat^{m}CT^{m}C-3 |

The following abbreviations are used throughout the examples, such as LNA (Locked nucleic acid oligonucleotide), BACC (2-[N,N′-di(2-guanidiniumpropyl)]aminoethylcholesterylcarbonate), 2-(Boc-oxyimino)-2-phenylacetatonitrile (BOC-ON), Chol (cholesterol), DIEA (diisopropylethylamine), DMAP (4-N,N-dimethylamino-pyridine), DOPE (L-α-dioleoyl phosphatidylethanolamine, Avanti Polar Lipids, USA or NOF, Japan), DLS (Dynamic Light Scattering), DSPC (1,2-distearoyl-sn-glycero-3-phosphocholine) (NOF, Japan), DSPE-PEG (1,2-distearoyl-sn-glycero-3-phosphoethanolamine-N-(polyethylene glycol)2000 ammonium salt or sodium salt, Avanti Polar Lipids, USA and NOF, Japan), KD (knowndown), EPC (egg phosphatidylcholine, Avanti Polar Lipids, USA) and C16mPEG-Ceramide (N-palmitoylsphingosine-1-[succinyl(methoxypolyethylene glycol)2000, Avanti Polar Lipids, USA). Other abbreviations such as FAM (6-carboxyfluorescein), FBS (fetal bovine serum), GAPDH (glyceraldehyde-3-phosphate dehydrogenase), DMEM (Dulbecco's Modified Eagle's Medium), MEM (Modified Eagle's Medium), TEAA (tetraethylammonium acetate), TFA (trifluoroacetic acid), RT-qPCR (reverse transcription-quantitative polymerase chain reaction) were also used.

¹H NMR spectra were obtained at 300 MHz and ¹³C NMR spectra at 75.46 MHz using a Varian Mercury 300 NMR spectrometer and deuterated chloroform as the solvents unless otherwise specified. Chemical shifts (d) are reported in parts per million (ppm) downfield from tetramethylsilane (TMS).

### Example 1

### Preparation of Bis[3-(Boc-amino)propyl]amine (Compound 1)

A solution of N-(3-aminopropyl)-1,3-propanediamine (1.45 g, 11.05 mmol) in 50 mL of anhydrous THF was stirred vigorously in an ice bath for 20 minutes. BOC-ON (5.998 g, 24.36 mmol) in 20 mL of anhydrous THF was added to the solution slowly over 2 hours. After the addition was complete, the ice bath was removed and the reaction mixture was stirred at room temperature for another 45 minutes. Then the reaction mixture was concentrated under reduced pressure. Compound 1 was obtained by column chromatography (ethyl acetate/methanol=from 4:1 to 3:2, v/v) with a yield of 57%: ¹H NMR 5.18, 3.23-3.17, 2.67-2.63, 1.68-1.60, 1.44; ¹³C NMR 155.9, 78.99, 47.51, 39.04, 29.87, 28.51.

### Example 2

### Preparation of 2-[Bis(3-N-Boc-aminopropyl)]amino alcohol (Compound 2)

To a 100 mL round-bottom flask were added bis[3-(Boc-amino)propyl]amine (compound 1, 2 g, 6 mmol), LiClO₄ (0.64 g, 6 mmol) and CH₃CN (24 mL). After the dissolution was complete, the flask was transferred to an ice-bath and 2 mL of ethylene oxide was added. The flask was then sealed and the reaction mixture was stirred at room temperature for 24 hours. After LiClO₄ was filtered, the reaction mixture was concentrated under reduced pressure and diluted with 100 mL of water. The crude product was obtained by extraction with ethyl ether (30 mL×3). The combined organic layer was washed with brine and dried over anhydrous sodium sulfate. Compound 2 was obtained after concentration in vacuo and purification by column chromatography (ethyl acetate/methanol=4/1, v/v) with a yield of 72%: ¹H NMR 5.05, 3.60-3.56, 3.20-2.14, 2.56-2.46, 1.68-1.60, 1.44; ¹³C NMR 155.99, 79.13, 58.98, 56.00, 51.63, 38.93, 28.48, 27.38.

### Example 3

### Preparation of 2-[Bis(3-N-Boc-aminopropyl)]aminoethylcholesteryl carbonate (Compound 3)

To a 250 mL round-bottom flask were added 2-[bis(3-N-Boc-aminopropyl)]amino alcohol (compound 2, 3.2 g, 8.5 mmol), DMAP (3.13 g, 25.6 mmol) and 100 mL of anhydrous methylene chloride. After the dissolution was complete, the reaction mixture was cooled to 0° C. in an ice-bath. Cholesteryl chloroformate (11.48 g, 25.6 mmol) was added and the reaction mixture was stirred for 4 hours in the ice bath and then for about 20 hours at room temperature. Thereafter, the solvent was removed in vacuo. The residue was dissolved in 100 mL of anhydrous ether and filtered. The filtrate was concentrated in vacuo and Compound 3 was recovered after purification by column chromatography (ethyl acetate) as a white solid with a yield of 72%.

### Example 4

### Preparation of 2-[Bis(3-aminopropyl)]aminoethylcholesteryl carbonate.2HCl (Compound 4)

2-[Bis(3-N-Boc-aminopropyl)]aminoethylcholesteryl carbonate (compound 3, 5.0 g, 6.34 mmol) was dissolved in 30 mL of anhydrous dioxane in a 100 mL round-bottom flask. To the solution was added 30 mL of 2M HCl solution in dioxane and the reaction mixture was stirred at room temperature for about one hour. After the reaction was complete, the reaction mixture was concentrated in vacuo to obtain a yellowish powder residue. The residue was washed three times with ether and dried under vacuum to give compound 4 with a yield of 98%.

### Example 5

### Preparation of 2-[Bis(3-guanidiniumpropyl)]aminoethylcholesteryl carbonate (Compound 5)

2-[Bis(3-aminopropyl)]aminoethylcholesteryl carbonate.2HCl (compound 4, 1.0 g, 1.43 mmol), 1H-pyrazole-1-carboxamidine.HCl (0.446 g, 3.04 mmol) and DIEA (1.00 g, 7.7 mmol) were placed into a 250 mL round bottom flask and 100 mL of anhydrous methylene chloride was added to the mixture. The reaction mixture was stirred at room temperature for 24 hours. After the reaction was complete, 300 mL of anhydrous ether was added to precipitate a white solid from the solution. Compound 5 was obtained as a white solid by washing the solid with ether and hexane alternatively three times. The yield was 68%.

### Example 6

### Preparation of Nanoparticles

In this example, nanoparticle compositions encapsulating various nucleic acids such as LNA-containing oligonucleotides were prepared. For example, compound 5, DOPE, Chol, DSPE-PEG and C₁₆mPEG-Ceramide were mixed at a molar ratio of 18:60:20:1:1 in 10 mL of 90% ethanol (total lipid 30 μmole). LNA oligonucleotides (0.4 μmole) were dissolved in 10 mL of 20 mM Tris buffer (pH 7.4-7.6). After being heated to 37° C., the two solutions were mixed together through a duel syringe pump and the mixed solution was subsequently diluted with 20 mL of 20 mM Tris buffer (300 mM NaCl, pH 7.4-7.6). The mixture was incubated at 37° C. for 30 minutes and dialyzed in 10 mM PBS buffer (138 mM NaCl, 2.7 mM KCl, pH 7.4). Stable particles were obtained after the removal of ethanol from the mixture by dialysis. The nanoparticle solution was concentrated by centrifugation. The nanoparticle solution was transferred into a 15 mL centrifugal filter device (Amicon Ultra-15, Millipore, USA). Centrifuge speed was at 3,000 rpm and temperature was at 4° C. during centrifugation. The concentrated suspension was collected after a given time and was sterilized by filtration through a 0.22 μm syringe filter (Millex-GV, Millipore, USA). A homogeneous suspension was obtained.

The diameter and polydispersity of nanoparticle were measured at 25° in water (Sigma) as a medium on a Plus 90 Particle Size Analyzer Dynamic Light Scattering Instrument (Brookhaven, N.Y.).

Encapsulation efficiency of LNA oligonucleotides was determined by UV-VIS (Agilent 8453). The background UV-vis spectrum was obtained by scanning solution, which was a mixed solution composed of PBS buffer saline (250 μL), methanol (625 μL) and chloroform (250 μL). In order to determine the encapsulated nucleic acids concentration, methanol (625 μL) and chloroform (250 μL) were added to PBS buffer saline nanoparticle suspension (250 μL). After mixing, a clear solution was obtained and this solution was sonicated for 2 minutes before measuring absorbance at 260 nm. The encapsulated nucleic acid concentration and loading efficiency was calculated according to equations (1) and (2):

Cₑₙ (μg/ml)=A₂₆₀×OD₂₆₀ unit (μg/mL)×dilution factor (μL/μL) (1)

where the dilution factor is given by the assay volume (μL) divided by the sample stock volume (μL).

Encapsulation efficiency (%)=[Cₑₙ/Cᵢₙᵢₜᵢₐₗ]×100 (2)

where Cₑₙ is the nucleic acid (i.e., LNA oligonucleotide) concentration encapsulated in nanoparticle suspension after purification, and Cᵢₙᵢₜᵢₐₗ is the initial nucleic acid (LNA oligonucleotide) concentration before the formation of the nanoparticle suspension.

The particle size, polydispersity and nucleic acid (LNA oligonucleotide) loading efficiency of various nanoparticle compositions are summarized in Tables 5 and 6. It is shown that these nanoparticle compositions achieved high nucleic acid loading efficiency (79-87%) with a size below 100 nm of nanoparticles with a low polydispersity.

TABLE 5

| | | | | | | Oligo |
|---|---|---|---|---|---|---|
| | | | | Particle | | Loading |
| Sample | Nanoparticle | | | Size | Poly- | Efficiency |
| No. | Composition | Molar Ratio | Oligo | (nm) | dispersity | (%) |
| 1 | Compd 5:DOPE: | 15:15:20:40:10 | Oligo-1 | 68 | 0.178 | 85 |
| | DSPC:Chol:PEG- | | | | | |
| | DSPE | | | | | |
| 2 | Compd 5:DOPE: | 15:5:20:50:10 | Oligo-1 | 95 | 0.199 | 86 |
| | DSPC:Chol:PEG- | | | | | |
| | DSPE | | | | | |
| 3 | Compd 5:DOPE: | 25:15:20:30:10 | Oligo-1 | 96 | 0.19 | 79 |
| | DSPC:Chol:PEG- | | | | | |
| | DSPE | | | | | |
| 4 | Compd 5:EPC:Chol: | 20:47:30:3 | Oligo-1 | 59.5 | 0.149 | 85 |
| | PEG-DSPE | | | | | |
| 5 | Compd 5:DOPE: | 17:60:20:3 | Oligo-1 | 76 | 0.135 | 80 |
| | Chol:PEG-DSPE | | | | | |
| 6 | Compd 5:DOPE: | 20:78:2 | Oligo-1 | 93 | 0.036 | 83 |
| | PEG-DSPE | | | | | |
| 7 | Compd 5:DOPE: | 17:60:20:3 | Oligo-2 | 66 | 0.155 | 87 |
| | Chol:C16mPEG- | | | | | |
| | Ceramide | | | | | |
| 8 | Compd 5:DOPE: | 18:60:20:1:1 | Oligo-2 | 80 | 0.129 | 82 |
| | Chol:PEG-DSPE: | | | | | |
| | C16mPEG-Ceramide | | | | | |

TABLE 6

| | | | | Particle | Zeta | | Oligo |
|---|---|---|---|---|---|---|---|
| Sample | Nanoparticle | | | Size | Potential | Poly- | Conc. |
| No. | Composition | Molar Ratio | Oligo | (nm) | (mV) | dispersity | (mg/mL) |
| NP1 | Compd 5:DOPE: | 18:60:20:1:1 | Oligo-2 | 79.9 | +24 | 0.125 | 1.6 |
| | Chol:PEG-DSPE: | | | | | | |
| | C16mPEG-Ceramide | | | | | | |
| NP2 | Compd 5:DOPE: | 18:60:20:1:1 | Scrambled | 84.6 | +21 | 0.092 | 1.57 |
| | Chol:PEG-DSPE: | | Oligo-2 | | | | |
| | C16mPEG-Ceramide | | (=Oligo-3) | | | | |
| NP3 | Compd 5:DOPE: | 18:60:20:1:1 | FAM- | 85.6 | +22 | 0.073 | 1.75 |
| | Chol:PEG-DSPE: | | Oligo-2 | | | | |
| | C16mPEG-Ceramide | | | | | | |
| NP4 | Compd 5:DOPE: | 18:60:20:1:1 | none | 77.9 | +38 | 0.243 | 0 |
| | Chol:PEG-DSPE: | | | | | | |
| | C16mPEG-Ceramide | | | | | | |

### Example 7

### Nanoparticle Stability

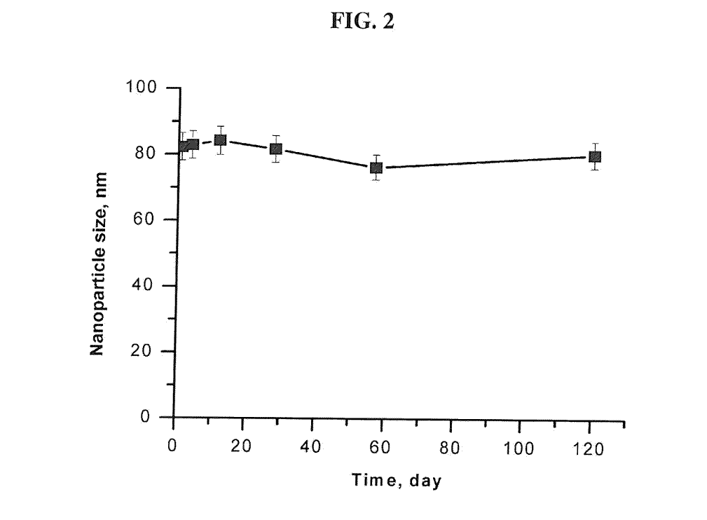

Nanoparticle stability was defined as their capability to retain the structural integrity in PBS buffer at 4° C. over time. The colloidal stability of nanoparticles was evaluated by monitoring changes in the mean diameter over time. Nanoparticles prepared by Sample No. NP1 in Table 6 were dispersed in 10 mM PBS buffer (138 mM NaCl, 2.7 mM KCl, pH 7.4) and stored at 4° C. At a given time point, about 20-50 μL of the nanoparticle suspension was taken and diluted with pure water up to 2 mL. The sizes of nanoparticles were measured by using Dynamic Light Scattering Technology (DLS) at 25° C. The results showed that there was almost no change in the particle sizes of the nanoparticles of Sample No. 8 when observed over 120 days. The results are shown in FIG. 2. The results showed that the nanoparticles containing the cationic lipid described herein (compound 5) as a component of the lipid carriers were very stable at 4° C. for a substantially prolonged period of time. The nanoparticles of Sample Nos. NP101, NP102, NP103 and NP104 (Table 7) also showed similar stability, as shown in FIG. 2.

TABLE 7

| | | | | Particle | | Oligo |
|---|---|---|---|---|---|---|
| Sample | Nanoparticle | | | Size | Poly- | Conc. |
| No. | Composition | Molar Ratio | Oligo | (nm) | dispersity | (μg/mL) |
| NP101 | Compd 5:DOPE:Chol: | 17:60:20:3 | Oligo-2 | 66.5 | 0.155 | 103.2 |
| | C16mPEG-Ceramide | | | | | |
| NP102 | Compd 5:DOGP:Chol: | 17:60:20:3 | Oligo-2 | 64.2 | 0.183 | 104.3 |
| | C16mPEG-Ceramide | | | | | |
| NP103 | Compd 5:DOPE:Chol: | 18:60:20:1:1 | Oligo-2 | 77.7 | 0.103 | 105.5 |
| | PEG-DSPE:C16mPEG- | | | | | |
| | Ceramide | | | | | |
| NP103 | Compd 5:DOGP:Chol: | 18:60:20:1:1 | Oligo-2 | 72.2 | 0.98 | 104.2 |
| | PEG-DSPE:C16mPEG- | | | | | |
| | Ceramide | | | | | |

### Example 8

### In Vitro Nanoparticle Cellular Uptake

The efficiency of cellular uptake of nucleic acids (LNA oligonucleotide Oligo-2) encapsulated in the nanoparticle described herein was evaluated in human prostate cancer cells (15PC3 cell line). Nanoparticles of Sample No. NP3 were prepared using the method described in Example 6. LNA oligonucleotides (Oligo-2) were labeled with FAM for fluorescent microscopy studies.

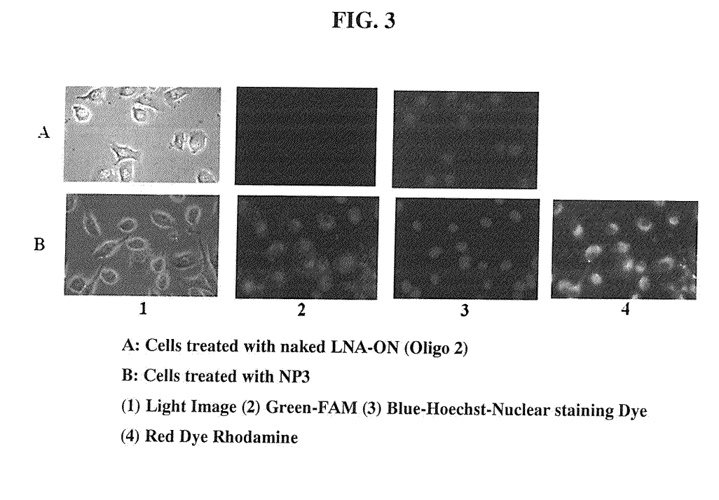

The nanoparticles were evaluated in the 15PC3 cell line. The cells were maintained in a complete medium (DMEM, supplemented with 10% FBS). A 12 well plate containing 2.5×10⁵ cells in each well was incubated overnight at 37° C. The cells were washed once with Opti-MEM and 400 mL of Opti-MEM was added to each well. Then, the cells were treated with a nanoparticle solution of Sample No. NP3 (200 nM) encapsulating nucleic acids (FAM-modified Oligo 2) or a solution of free nucleic acids without the nanoparticles (naked FAM-modified Oligo 2) as a control. The cells were incubated for 24 hours at 37° C. The cells were washed with PBS five times, and then stained with 300 mL of Hoechst solution (2 mg/mL) per well for 30 minutes, followed by washing with PBS 5 times. The cells were fixed with pre-cooled (−20° C.) 70% EtOH at −20° C. for 20 minutes. The cells were inspected under fluorescent microscope and the images are shown in FIG. 3.

The cells treated with the free nucleic acids under the same condition didn't show any cellular uptake of nucleic acids as shown in FIG. 3A. The cells incubated with the nanoparticles had a significant nuclear accumulation of the nucleic acids (FIG. 3B). In addition, the cells treated with the nanoparticles showed a large diffuse cytoplasmic localization of the nucleic acids. A few additional cytoplasmic punctuate accumulation patterns of the nucleic acids have also been observed, which is typical for endocytic vesicles as shown in FIG. 3B. The cells treated with nanoparticles of Sample No. NP105 (Table 8) also showed cellular uptake of nucleic acids similarly as shown in FIG. 3.

TABLE 8

| | | | | | | Oligo |
|---|---|---|---|---|---|---|
| Sample | Nanoparticle | | | Particle | | Conc. |
| No. | Composition | Molar Ratio | Oligo | Size (nm) | Polydispersity | (μg/mL) |
| NP105 | Compd 5:DOPE: | 17:60:20:3 | FAM- | 78.3 | 0.12 | 132 |
| | Chol:PEG-DSPE | | Oligo-2 | | | |

The results showed that the nanoparticles encapsulating nucleic acids crossed the cell membranes without the aid of transfection agents and accumulated in the nucleus and cytoplasm. The nanoparticle described herein provides a means to deliver nucleic acids inside the cells, preferably tumor cells.

### Example 9

### In Vitro Efficacy of Nanoparticles on mRNA Down-Regulation in Human Epidermal Cancer Cells

The efficacy of Sample No. NP5 was evaluated in human epidermal cancer cells (A431 cell line). The A431 cells overexpress epidermal growth factor receptors (EGFR). The cells were treated with nanoparticles encapsulating antisense ErbB3 oligonucleotides (Sample NP5). The cells were also treated with nanoparticles encapsulating oligonucleotides with a scrambled sequence (Sample No. NP6) or empty placebo nanoparticles (Sample No. NP7) as a control. The nanoparticles were prepared using the method described in Example 6 (Table 9).

TABLE 9

| Sample | Nanoparticle | Molar | | Particle | Poly- | Oligo Conc. |
|---|---|---|---|---|---|---|
| No. | Composition | Ratio | Oligo | Size (nm) | dispersity | (μg/mL) |
| NP5 | Compd 5:DOPE: | 18:60:20:1:1 | Oligo-2 | 80 | 0.129 | 129.5 |
| | Chol:PEG-DSPE: | | | | | |
| | C16mPEG- | | | | | |
| | Ceramide | | | | | |
| NPS | Compd 5:DOPE: | 18:60:20:1:1 | Oligo-3 | 85.5 | 0.197 | 139.1 |
| | Chol:PEG-DSPE: | | | | | |
| | C16mPEG- | | | | | |
| | Ceramide | | | | | |
| NP7 | Compd 5:DOPE: | 18:60:20:1:1 | none | 77.9 | 0.243 | 0 |
| | Chol:PEG-DSPE: | | | | | |
| | C16mPEG- | | | | | |
| | Ceramide | | | | | |

The cells were maintained in a complete medium (F-12K or DMEM, supplemented with 10% FBS). A 12 well plate containing 2.5×10⁵ cells in each well was incubated overnight at 37° C. The cells were washed once with Opti-MEM® and 400 μL of Opti-MEM® was added per each well. Then, the cells were treated with nanoparticles of Sample Nos. NP5, NP6 or NP7. The cells were incubated for 4 hours, followed by addition of 600 μL of media per well, and incubation for 24 hours. After 24 hours of the treatment, the intracellular mRNA levels of the target gene such as human ErbB3, and a housekeeping gene such as GAPDH were measured by RT-qPCR. The expression levels of ErbB3 mRNA genes were normalized to that of GAPDH.

For the mRNA down-regulation study, the total RNA was prepared by using RNAqueous Kit® (Ambion) following the manufacturer's instruction. The RNA concentrations were determined by OD_{260 nm} using Nanodrop. All reagents were purchased from Applied Biosystems: High Capacity cDNA Reverse Transcription Kit® (Cat. No. 4368813), 20×PCR master mix (Cat. No. 4304437), and TaqMan® Gene Expression Assays kits for human GAPDH (Cat. No. 0612177).

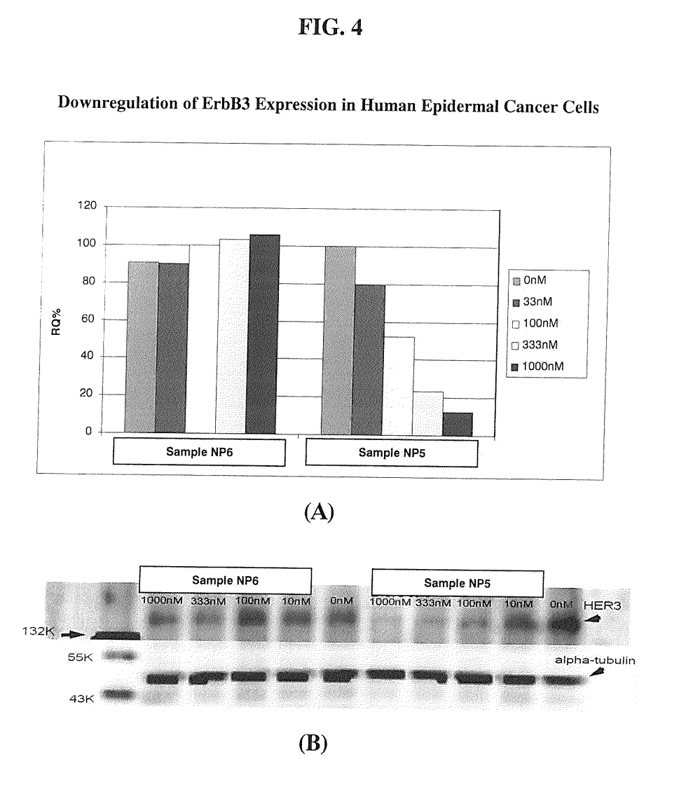

The nanoparticles encapsulating antisense ErbB3 oligonucleotides (Sample No. NP5) showed dose-dependent mRNA knockdown with IC₅₀ as low as 100 nM (FIG. 4A) in human epidermal cancer cells. This mRNA knockdown was correlated with the ErbB3 protein levels (FIG. 4B). The down-regulation of ErbB3 expression was confirmed by measuring the ErbB3 protein levels from the cells by the Western Blot method. Anti-ErbB3 antibody was purchased from Santa Cruz (SC285) and applied. The nanoparticles encapsulating scrambled oligonucleotides (Sample No. NP6) did not inhibit ErbB3 expression.

The results showed that nanoparticles encapsulating antisense oligonucleotides inhibit target gene expression selectively and in a dose-dependent manner. The nanoparticles described herein provide a means for inhibiting target gene expression in the absence of transfection agents.

### Example 10

### In Vitro Efficacy of Nanoparticles on mRNA Down-Regulation in Human Gastric Cancer Cells

The efficacy of the nanoparticles described herein was evaluated in human gastric cancer cells (N87cell line). The cells were treated with one of the following: nanoparticles encapsulating antisense ErbB3 oligonucleotides (Sample NP5), nanoparticles encapsulating oligonucleotides with a scrambled sequence (Sample No. NP6) or empty placebo nanoparticles (Sample No. NP7). The in vitro efficacy of each of the nanoparticles on downregulation of ErbB3 expression was measured by the procedures described in Example 9.

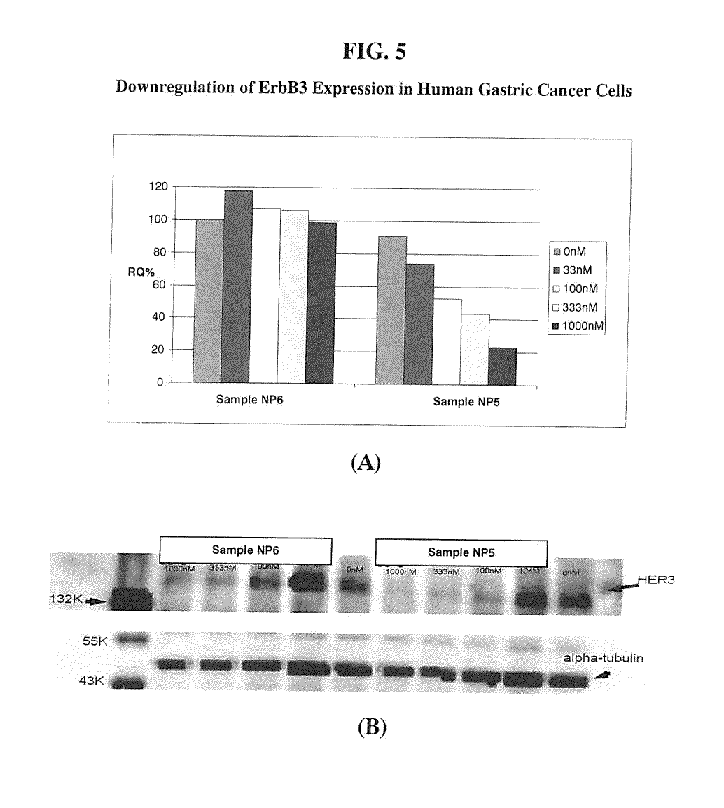

The nanoparticles encapsulating antisense oligonucleotides inhibited target gene or protein expression dose-dependently in human gastric cancer cells. The inhibition was sequence specific. The scrambled oligonucleotides did not inhibit the target ErbB3 gene or protein expression. The results are shown in FIG. 5.

### Example 11

### In Vitro Efficacy of Nanoparticles on mRNA Down-Regulation in Human Lung Cancer Cells

The efficacy of the nanoparticles described herein was also evaluated in human lung cancer cells (A549 cell line). The cells were treated with one of the following: nanoparticles encapsulating antisense ErbB3 oligonucleotides (Sample NP5), nanoparticles encapsulating oligonucleotides with a scrambled sequence (Sample No. NP6) or empty placebo nanoparticles (Sample No. NP7). The in vitro efficacy of each of the nanoparticles on downregulation of ErbB3 expression was measured by the procedures described in Example 9.

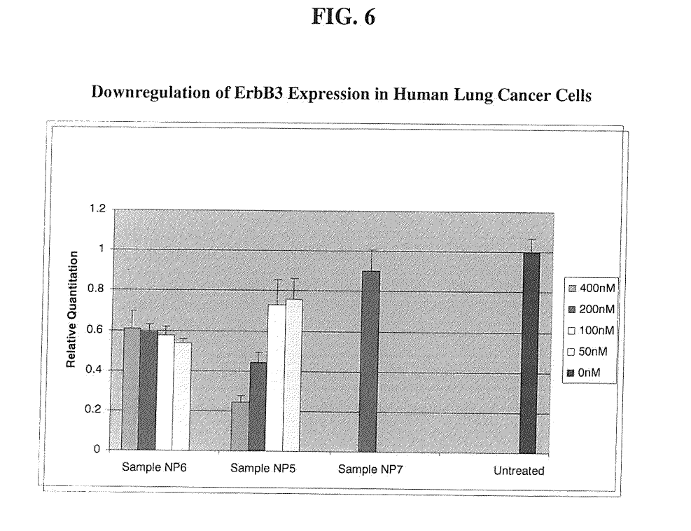

The nanoparticles encapsulating antisense oligonucleotides inhibited target gene or protein expression dose-dependently in human lung cancer cells. The results showed IC₅₀ of about 200 nM in the cancer cells. The inhibition was sequence specific. The scrambled oligonucleotides did not inhibit the target ErbB3 gene or protein expression. The results are shown in FIG. 6.

### Example 12

### In Vitro Efficacy of Nanoparticles on mRNA Down-Regulation in Human Prostate Cancer Cells

The efficacy of the nanoparticles described herein was also evaluated in human prostate cancer cells (15PC3 cell line). The cells were treated with one of the following: nanoparticles encapsulating antisense ErbB3 oligonucleotides (Sample NP5), nanoparticles encapsulating oligonucleotides with a scrambled sequence (Sample No. NP6) or empty placebo nanoparticles (Sample No. NP7). The in vitro efficacy of each of the nanoparticles on downregulation of ErbB3 expression was measured by the procedures described in Example 9.

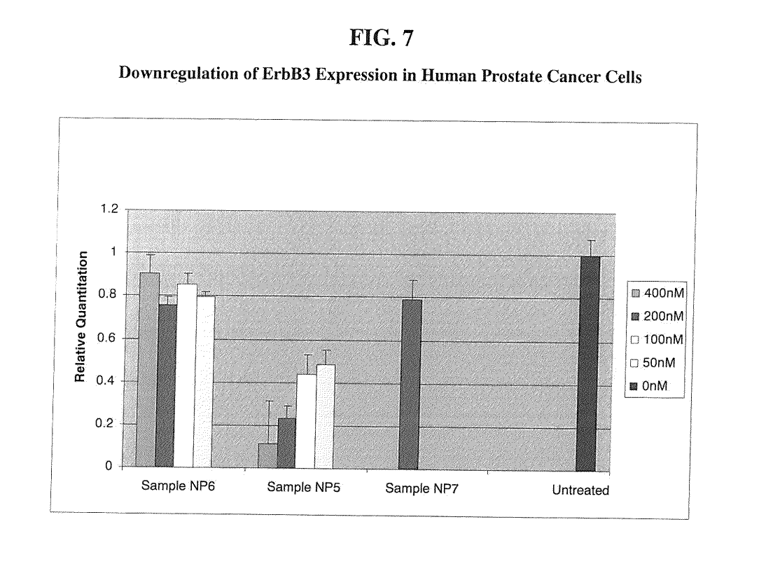

The nanoparticles encapsulating antisense oligonucleotides inhibited target gene or protein expression dose-dependently with IC₅₀ of about 100 nM in human prostate cancer cells. The inhibition was sequence specific. The scrambled oligonucleotides did not inhibit the target ErbB3 gene or protein expression. The results are shown in FIG. 7.

### Example 13

### In Vitro Efficacy of Nanoparticles on mRNA Down-Regulation in Human Breast Cancer Cells

The efficacy of the nanoparticles described herein was also evaluated in human breast cancer cells (MCF7 cell line). The cells were treated with one of the following: nanoparticles encapsulating antisense ErbB3 oligonucleotides (Sample NP5), nanoparticles encapsulating oligonucleotides with a scrambled sequence (Sample No. NP6) or empty placebo nanoparticles (Sample No. NP7). The in vitro efficacy of each of the nanoparticles on downregulation of ErbB3 expression was measured by the procedures described in Example 9.

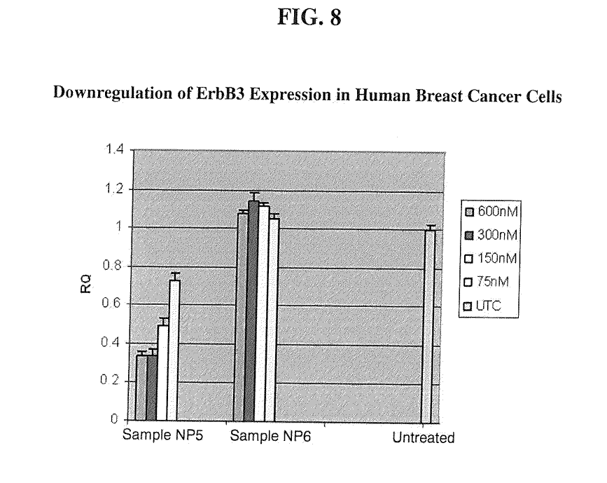

The nanoparticles encapsulating antisense oligonucleotides inhibited target gene or protein expression dose-dependently in human breast cancer cells. The results showed about IC₅₀ of 150 nM in the cancer cells. The inhibition was sequence specific. The scrambled oligonucleotides did not inhibit the target ErbB3 gene or protein expression. The results are shown in FIG. 8.

### Example 14

### In Vitro Efficacy of Nanoparticles on mRNA Down-Regulation in Human KB Cancer Cells

The efficacy of the nanoparticles described herein was also evaluated in human KB cancer cells (KB cell line). The cells were treated with one of the following: nanoparticles encapsulating antisense ErbB3 oligonucleotides (Sample NP5), nanoparticles encapsulating oligonucleotides with a scrambled sequence (Sample No. NP6) or empty placebo nanoparticles (Sample No. NP7). The in vitro efficacy of each of the nanoparticles on downregulation of ErbB3 expression was measured by the procedures described in Example 9.

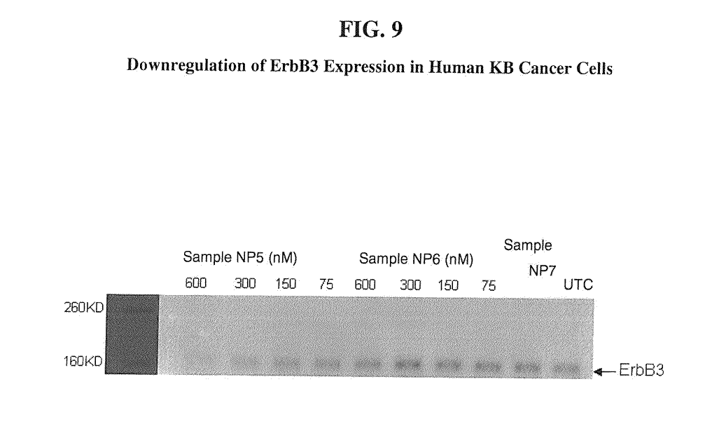

The nanoparticles encapsulating antisense oligonucleotides inhibited target gene or protein expression dose-dependently in human KB cancer cells. The inhibition was sequence specific. The scrambled oligonucleotides did not inhibit the target ErbB3 gene or protein expression. The results are shown in FIG. 9.

### Example 15

### In Vitro Efficacy of Nanoparticles on mRNA Down-Regulation in Human Prostate Cancer Cells

The efficacy of the nanoparticles described herein was also evaluated in another type of human prostate cancer cells (DU145 cell line). The cells were treated with each of nanoparticles encapsulating antisense ErbB3 oligonucleotides (Sample NP5), nanoparticles encapsulating oligonucleotides with a scrambled sequence (Sample No. NP6) or empty placebo nanoparticles (Sample No. NP7). The in vitro efficacy of each of the nanoparticles on downregulation of ErbB3 expression was measured by the procedures described in Example 9.

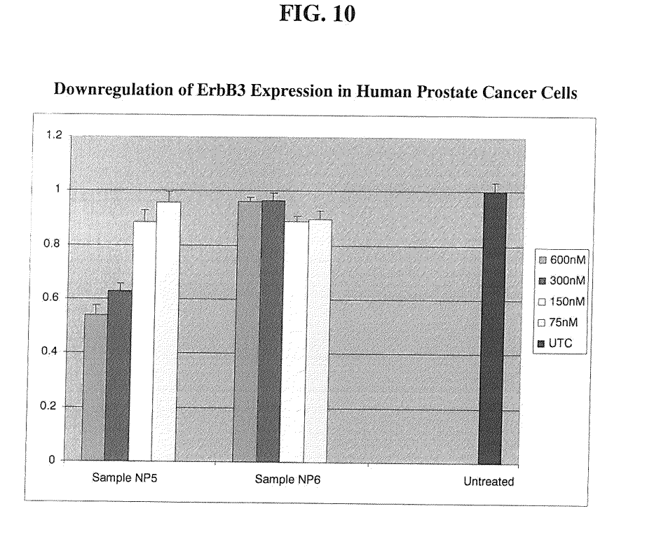

The nanoparticles encapsulating antisense oligonucleotides inhibited target gene or protein expression dose-dependently in human prostate cancer cells. The inhibition was sequence specific. The scrambled oligonucleotides did not inhibit the target ErbB3 gene or protein expression. The results are shown in FIG. 10.

The nanoparticles described herein delivered nucleic acids into a variety of cancer cells such as human lung, prostate, breast, and KB cancer cells. As described in FIGS. 6-10, the mRNA KD efficacies in the cancer cell lines range from about 50 to about 400 nM of antisense oligonucleotides encapsulated in the nanoparticles in the order of 15PC3>MCF7≈A431≈N87>A549>DU145≈KB. The mRNA KD was correlated with the protein KD in each of the tested cancer cells.

### Example 16

### In Vivo Efficacy of Nanoparticles on mRNA Down-Regulation in Tumor and Liver of Human Prostate Cancer Xenografted Mice Model

The in vivo efficacy of nanoparticles described herein was evaluated in human prostate cancer xenografted mice. The 15PC3 human prostate tumors were established in nude mice by subcutaneous injection of 5×10⁶ cells/mouse into the right auxiliary flank. When tumors reached the average volume of 100 mm³, the mice were randomly grouped 5 mice per group. The mice of each group were treated with nanoparticle encapsulating antisense ErbB3 oligonucleotides (Sample NP5) or corresponding naked oligonucleotides (Oligo 2). The nanoparticles were given intravenously (i.v.) at 15 mg/kg/dose, 5 mg/kg/dose, 1 mg/kg/dose, or 0.5 mg/kg/dose at q3d×4 for 12 days. The dosage amount is based on the amount of oligonucleotides in the nanoparticles. The naked oligonucleotides were given intraperitoneally (i.p.) at 30 mg/kg/dose or intravenously at 25 mg/kg/dose or 45 mg/kg/dose at q3d×4 for 12 days. The mice were sacrificed twenty four hours after the final dose. Plasma samples were collected from the mice and stored at −20° C. Tumor and liver samples were also collected from the mice. The samples were analyzed for mRNA KD.

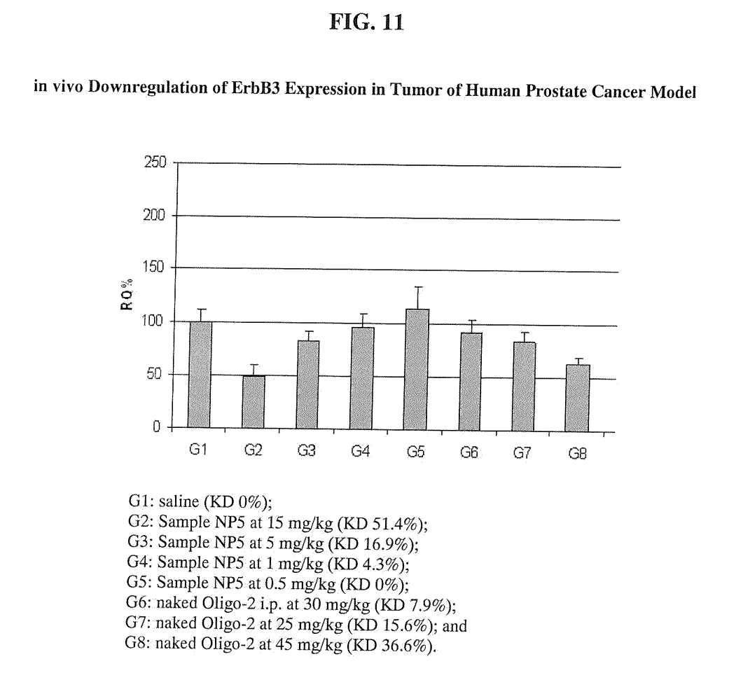

In the tumor samples of the mice treated with the nanoparticles, the treatment inhibited ErbB3 mRNA expression dose-dependently. The ErbB3 expression was inhibited over about 51% at the dose of 15 mg/kg (G2). In the tumor samples of the animals treated with the naked oligonucleotides, only about 37% of ErbB3 mRNA expression was inhibited at the dose of 45 mg/kg of oligo-2 (G8). The results are shown in FIG. 11.

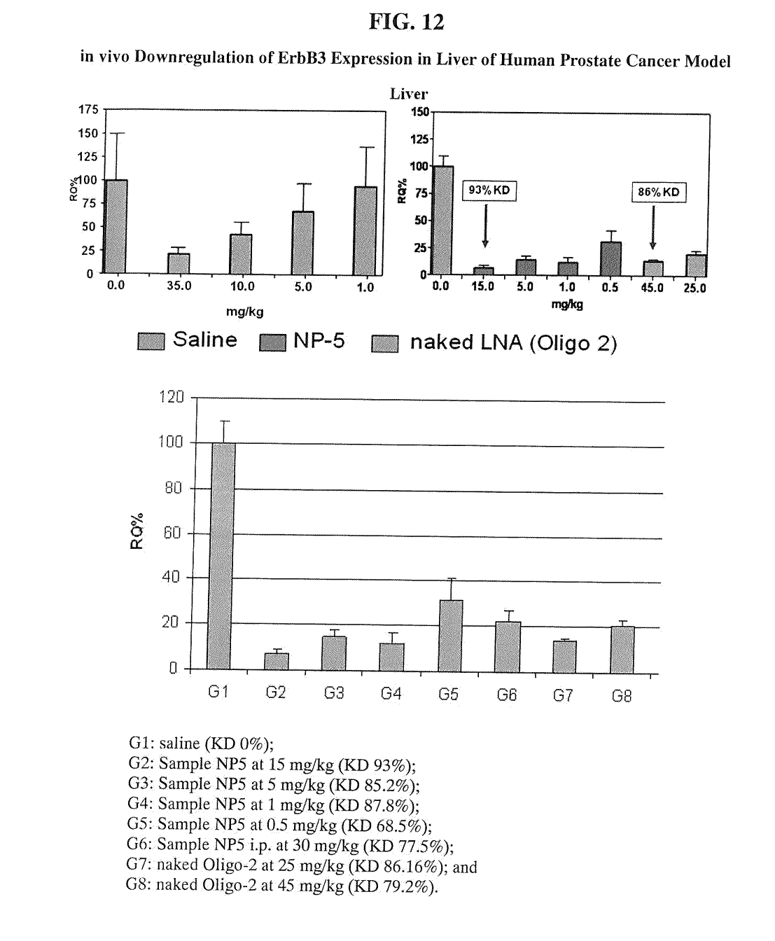

In the liver samples, the nanoparticles were very potent in the downregulation of the target gene expression at a low dose, as compared to the naked oligonucleotides. The nanoparticles showed about 93% KD activity at 15 mg/kg/dose (G2). The nanoparticles also showed about 87% KD activity at 1 mg/kg/dose (G4) which was as effective as 25 mg/kg/dose of Oligo-2 (G7). The results are shown in FIG. 12.

The results showed that the nanoparticles encapsulating antisense oligonucleotides inhibited expression of the target gene in both tumor and liver significantly and effectively, as compared to naked LNA oligonucleotides.

### Example 17

### In Vivo Efficacy of Nanoparticles on mRNA Down-Regulation in Human Colon Cancer Xenografted Mice Model

The in vivo efficacy of the nanoparticles described herein was evaluated in human colon cancer xenografted mice. The nanoparticles described herein (Sample NP5) were given via intratumoral injection to the mice with human DLD-1 tumors at q3d×4 for 12 days. The naked oligonucleotides (Oligo 2), scrambled oligonucleotides (Oligo 3), and nanoparticles containing scrambled oligonucleotides (Sample NP6) were also given to the mice. Tumor samples from the mice of each test group were collected and analyzed by using qRT-PCR for mRNA down-regulation.

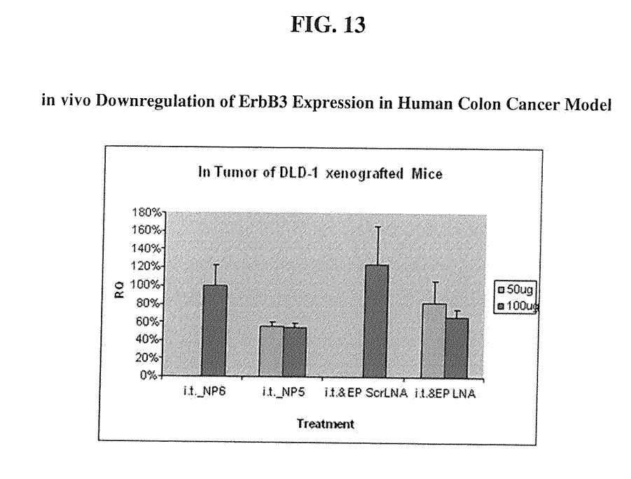

In the mice treated with the nanoparticles containing antisense ErbB3 oligonucleotides, the treatment inhibited ErbB3 mRNA expression significantly, as compared to the naked antisense oligonucleotides or the nanoparticles containing scrambled oligonucleotides. The results are shown in FIG. 13. The results showed that the nanoparticles encapsulating antisense oligonucleotides inhibited expression of the target gene in the tumor significantly and effectively, as compared to naked LNA oligonucleotides.

### Example 18

### In Vivo Efficacy of Nanoparticles on mRNA Down-Regulation in Human Cancer Xenografted Mice Model with Metastatis in Liver

The in vivo efficacy of the nanoparticles described herein was evaluated in human cancer xenografted mice with metastasis to the liver. The A549 cancer cells were injected intrasplenically, followed by a splenectomy to establish metastatic liver disease. Two days following the splenectomy, the mice of each group were intravenously given nanoparticles encapsulating antisense ErbB3 oligonucleotides (Sample NP5) or scrambled oligonucleotides (Sample NP6) at 0.5 mg/kg/dose at q3d×10. Naked antisense ErbB3 oligonucleotides (Oligo 2) were given intravenously at 35 mg/kg/dose at q3d×4. The survival of the animals was observed.

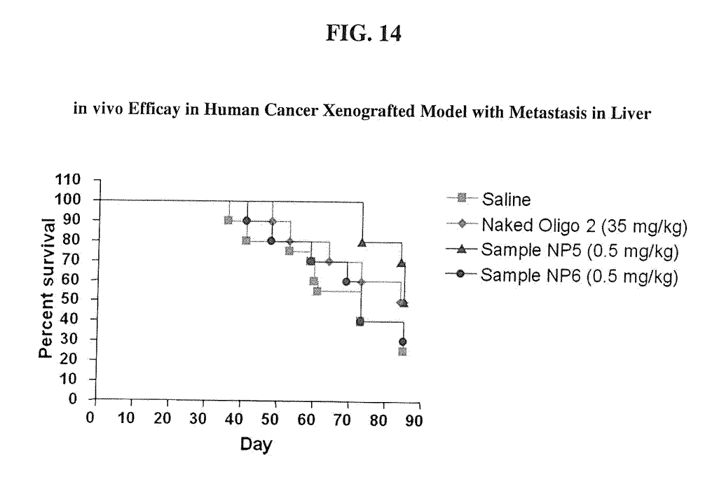

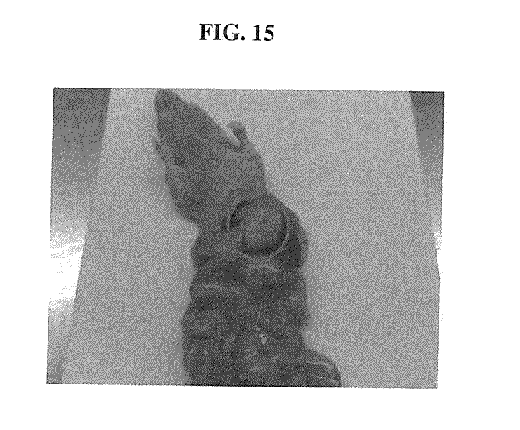

The treatment with the nanoparticles containing antisense ErbB3 oligonucleotides increased survival (about 85 days), as compared to about 73 days of the control animals. The results are shown in FIG. 14. Gross observation indicated that deaths of the animals were due to liver metastasis. An image of a representative animal with liver metastasis is shown in FIG. 15.

The results showed that the nanoparticles encapsulating antisense oligonucleotides improved metastatic cancer (i.e. metastatic cancer in the liver), as compared to naked LNA oligonucleotides.

We claim:

1. A nanoparticle composition comprising:
(i) a cationic lipid having Formula (I): 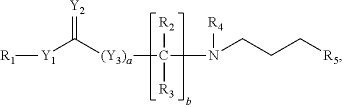
wherein
R₁ is a cholesterol or analog thereof;
Y₁ and Y₃ are independently O, S or NR₇;
Y₁ is O, S or NR₇;
(a) is 0 or 1;
R₂ and R₃ are independently hydrogen or lower alkyl;
(b) is a positive integer from about 2 to about 10;
R₄ is hydrogen, lower alkyl or 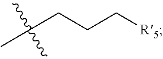
R₅ is 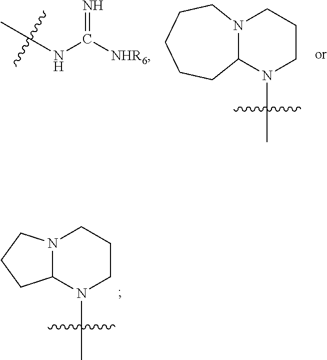
R′₅ is NH₂, 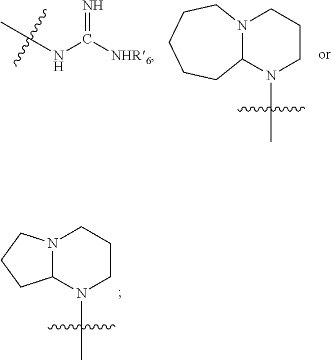
R₆, R′₆ and R₇ are independently hydrogen or lower alkyl;
(ii) a fusogenic lipid; and
(iii) a PEG lipid.

2. The nanoparticle composition of claim 1, wherein R₄ is 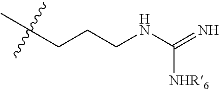
and
R₅ is

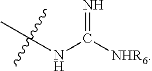

3. The nanoparticle composition of claim 2, wherein R₆ and R′₆ are hydrogen.

4. The nanoparticle composition of claim 1, wherein Y₁, Y₂ and Y₃ are all oxygen.

5. The nanoparticle composition of claim 1, wherein (a) is 1 and (b) is 2.

6. The nanoparticle composition of claim 1, wherein both R₂ and R₃ are hydrogen.

7. The nanoparticle composition of claim 1, wherein the cationic lipid is

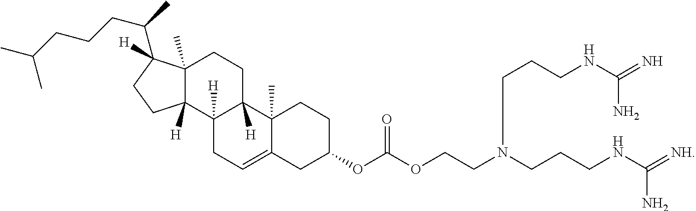

8. The nanoparticle composition of claim 1, wherein the fusogenic lipid is selected from the group consisting of DOPE, DOGP, POPC, DSPC, EPC, and combinations thereof.

9. The nanoparticle composition of claim 1, wherein the PEG lipid is selected from the group consisting of PEG-DSPE, PEG-dipalmitoylglycamide, C16mPEG-ceramide and combinations thereof.

10. The nanoparticle composition of claim 1, further comprising cholesterol.

11. The nanoparticle composition of claim 1, wherein the cationic lipid has a molar ratio ranging from about 10% to about 99.9% of the total lipid present in the nanoparticle composition.

12. The nanoparticle composition of claim 11, wherein the cationic lipid has a molar ratio ranging from about 15% to about 25% of the total lipid present in the nanoparticle composition.

13. The nanoparticle composition of claim 1, wherein a molar ratio of a cationic lipid, a non-cholesterol-based fusogenic lipid, a PEG lipid and cholesterol is about 15-25%:20-78%:0-50%:2-10%:of the total lipid present in the nanoparticle composition.

14. The nanoparticle composition of claim 1 selected from the group of a mixture of:
a cationic lipid of Formula (I), a diacylphosphatidylethanolamine, a PEG conjugated to phosphatidylethanolamine (PEG-PE), and cholesterol;
a cationic lipid of Formula (I), a diacylphosphatidylcholine, a PEG conjugated to phosphatidylethanolamine (PEG-PE), and cholesterol;
a cationic lipid of Formula (I), a diacylphosphatidylethanolamine, a diacylphosphatidylcholine, a PEG conjugated to phosphatidylethanolamine (PEG-PE), and cholesterol;
a cationic lipid of Formula (I), a diacylphosphatidylethanolamine, a PEG conjugated to ceramide (PEG-Cer), and cholesterol; and
a cationic lipid of Formula (I), a diacylphosphatidylethanolamine, a PEG conjugated to phosphatidylethanolamine (PEG-PE), a PEG conjugated to ceramide (PEG-Cer), and cholesterol.

15. A nanoparticle comprising nucleic acids encapsulated with the nanoparticle composition of claim 1.

16. The nanoparticle of claim 15, wherein the nucleic acids is a single stranded or double stranded oligonucleotide.

17. The nanoparticle of claim 15, wherein the nucleic acids is selected from the group consisting of deoxynucleotide, ribonucleotide, locked nucleic acids (LNA), short interfering RNA (siRNA), microRNA (miRNA), aptamers, peptide nucleic acid (PNA), phosphorodiamidate morpholino oligonucleotides (PMO), tricyclo-DNA, double stranded oligonucleotide (decoy ODN), catalytic RNA (RNAi), aptamers, spiegelmers, CpG oligomers and combinations thereof.

18. The nanoparticle of claim 16, wherein the oligonucleotide is an antisense oligonucleotide.

19. The nanoparticle of claim 16, wherein the oligonucleotide has phosphorothioate linkages.

20. The nanoparticle of claim 16, wherein the oligonucleotide includes LNA.

21. The nanoparticle of claim 16, wherein the oligonucleotide has from about 8 to 50 nucleotides.

22. The nanoparticle of claim 16, wherein the oligonucleotide inhibits expression of oncogenes, pro-angiogenesis pathway genes, pro-cell proliferation pathway genes, viral infectious agent genes, and pro-inflammatory pathway genes.

23. The nanoparticle of claim 16, wherein the oligonucleotide is selected from the group consisting of antisense HIF-1α oligonucleotides, antisense survivin oligonucleotides, antisense ErbB3 oligonucleotides, β-catenine oligonucleotides and antisense Bcl-2 oligonucleotides.

24. The nanoparticle of claim 15, wherein the charge ratio of the cationic lipid and the nucleic acids ranges from about 1:1 to about 20:1.

25. The nanoparticle of claim 15, wherein the nanoparticle has a size ranging from about 50 nm to about 150 nm.

26. The nanoparticle composition of claim 1, wherein the cationic lipid, DOPE, cholesterol, and C16mPEG-Ceramide is included in a molar ratio of about 17%:60%:20%:3% of the total lipid present in the nanoparticle composition, wherein the cationic lipid is

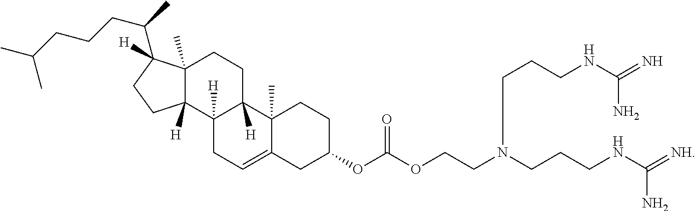

27. The nanoparticle composition of claim 1, wherein the nanoparticle contains the cationic lipid, DOPE, cholesterol, PEG-DSPE, and C16mPEG-Ceramide in a molar ratio of about 18%:60%:20%:1%:1% of the total lipid present in the nanoparticle composition, wherein the cationic lipid is

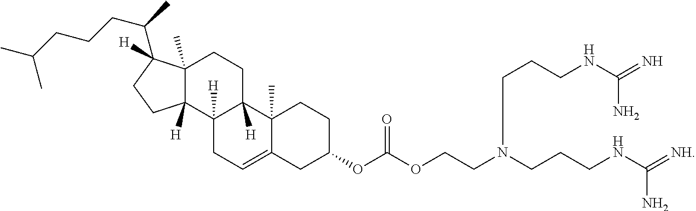

28. A method of introducing an oligonucleotide into a cell comprising:
contacting a cell with a nanoparticle of claim 15.

29. A method of inhibiting a gene expression in human cells or tissues, comprising:
contacting human cells or tissues with a nanoparticle of claim 15.

30. The method of claim 29, wherein the cells or tissues are cancer cells or tissues.

31. A method of downregulating a gene expression in a mammal, comprising:
administering an effective amount of a nanoparticle of claim 15 to a mammal in need thereof.

32. A method of inhibiting the growth or proliferation of cancer cells comprising:
contacting a cancer cell with a nanoparticle of claim 15.

33. The method of claim 32, further comprising administering a chemotherapeutic agent.

34. A method of treating a cancer in a mammal, comprising:
administering an effective amount of a nanoparticle of claim 15 to a mammal in need thereof.

35. The method of claim 34, wherein the cancer is metastatic into the liver.
